(19) United States
(12) Patent Application Publication
(10) Pub. No.: US 2024/0000500 A1
(43) Pub. Date: Jan. 4, 2024

(54) ADVANCED BIPOLAR SEAL QUALITY PREDICTION

(71) Applicant: Cilag GmbH International, Zug (CH)

(72) Inventors: Richard W. Timm, Cincinnati, OH (US); Anshu Gupta, Bridgewater, NJ (US); Shaun B. Schaeffer, Cincinnati, OH (US); Swapnil Rai, Lucknow (IN); Matjaz Jogan, Philadelphia, PA (US); Jeffrey D. Messerly, Cincinnati, OH (US); Jonathan H. Duff, Madison, WI (US); Rayonna M. Gordon, Providence Forge, VA (US); Benjamin M. Boyd, Fairborn, OH (US); Claire J. Dong, Durham, NC (US)

(21) Appl. No.: 18/202,604

(22) Filed: May 26, 2023

Related U.S. Application Data
(60) Provisional application No. 63/357,177, filed on Jun. 30, 2022.

(51) Int. Cl. A61B 18/14; A61B 90/00

(52) U.S. Cl. CPC A61B 18/1445; A61B 90/37; A61B 2018/1455; A61B 2017/320097

(57) ABSTRACT

A system for assessing the quality of seals applied to tissue by electrosurgical instruments is provided. The system can include a surgical device with a control circuit to determine that an end effector of the surgical device is in a closed configuration, determine a presence of tissue disposed between a first jaw and a second jaw of the end effector, monitor motion of the end effector in the closed configuration with tissue present between the first and second jaw, detect motion of the end effector outside of a predetermined range based on the motion data, and provide feedback data based on the detected motion of the end effector.

### CROSS-REFERENCE TO RELATED APPLICATIONS

This application claims the benefit of priority under 35 U.S.C. § 119(e) to U.S. Provisional Patent Application No. 63/357,177, titled ADVANCED BIPOLAR SEAL QUALITY PREDICTION, filed on Jun. 30, 2022, the disclosure of which is herein incorporated by reference in its entirety.

### BACKGROUND

The present disclosure relates to electrosurgical instruments that are designed to seal and cut tissue.

### SUMMARY

The following summary is provided to facilitate an understanding of some of the innovative features unique to the aspects disclosed herein, and is not intended to be a full description. A full appreciation of the various aspects can be gained by taking the entire specification, claims, and abstract as a whole.

In one general aspect, the present disclosure is directed to a surgical device. The surgical device comprises an end effector comprising a first jaw and a second jaw. The surgical device further comprises a first sensor to detect tissue disposed between the first jaw and the second jaw, a second sensor to detect the end effector in a closed configuration, and a third sensor to detect motion of the end effector. The surgical device further comprises a control circuit communicably coupled to the first sensor, the second sensor, and the third sensor. The control circuit comprises a processor and a memory, wherein the memory stores instructions that, when executed by the processor, cause the control circuit to determine that the end effector is in a closed configuration based on first sensor data, determine presence of tissue disposed between the first jaw and the second jaw based on second sensor data, and monitor motion of the end effector in the closed configuration with tissue present between the first and second jaw based on third sensor data. The memory stores further instructions that, when executed by the processor, cause the control circuit to detect motion of the end effector outside of a predetermined range based on the motion, and provide feedback data based on the detected motion of the end effector.

In at least one aspect, the memory stores further instructions that, when executed by the processor, cause the control circuit to calculate tension on the tissue based on the motion.

In at least one aspect, the feedback is visual. In at least one aspect, the visual feedback is superimposed over a display image of a surgical site.

In at least one aspect, the feedback is auditory.

In at least one aspect, the first jaw comprises a clamp arm and the second jaw comprises an ultrasonic blade.

In at least one aspect, the first jaw comprises an anvil and the second jaw comprises a staple cartridge.

In another general aspect, the present disclosure is directed to a surgical device. The surgical device comprises an end effector comprising a first jaw and a second jaw. The surgical device further comprises a first sensor to detect tissue disposed between the first jaw and the second jaw, a second sensor to detect that the end effector is in a closed configuration, a first fiducial mark, and a second fiducial mark. The surgical device further comprises a control circuit communicably coupled to the first sensor, the second sensor, and a camera. The control circuit comprises a processor and a memory, wherein the memory stores instructions that, when executed by the processor, cause the control circuit to receive video data of a surgical site from the camera, determine that the end effector is in the closed configuration on first sensor data, and determine presence of tissue disposed between the first jaw and the second jaw based on second sensor data. The memory stores further instructions that, when executed by the processor, cause the control circuit to determine a location of a device tip in the video data based on the first fiducial mark and the second fiducial mark, determine a region of interest in the video data based on the location of the device tip in the video data, and analyze the region of interest of the end effector in the closed configuration with tissue present between the first jaw and the second jaw. The memory stores further instructions that, when executed by the processor, cause the control circuit to determine tension on the tissue based on the analysis, and provide feedback based on the tension.

In at least one aspect, the memory stores further instructions that, when executed by the processor, cause the control circuit to detect motion of the end effector outside of a predetermined range based on the analysis.

In at least one aspect, the memory stores further instructions that, when executed by the processor, cause the control circuit to determine device type based on the first fiducial mark and the second fiducial mark.

In at least one aspect, the feedback is visual. In at least one aspect, the visual feedback is superimposed over a display image of a surgical site.

In at least one aspect, the feedback is auditory.

In at least one aspect, the first jaw comprises a clamp arm and the second jaw comprises an ultrasonic blade.

In at least one aspect, the first jaw comprises an anvil and the second jaw comprises a staple cartridge.

In yet another general aspect, the present disclosure is directed to a surgical system that includes a surgical instrument, an RF energy source, and a control circuit. The surgical instrument comprises an end effector to capture tissue. The end effector comprises an electrode to apply radio-frequency (RF) energy to the tissue captured by the end effector. The RF energy source is to provide RF energy to the electrode. The control circuit is to transmit a control signal to the RF energy source. The control signal causes the RF energy source to provide RF energy to the electrode to apply a seal to the tissue captured by the end effector. The control circuit is further to predict a quality of the seal and provide feedback to a user based on the prediction.

In at least one aspect, the control circuit is to generate a value associated with the seal and compare the value to a seal threshold. To provide feedback to the user based on the prediction, the control circuit is to provide feedback to the user based on results of the compare.

In at least one aspect, the control circuit is to abstain from providing feedback based on the value reaching or exceeding the seal threshold.

In at least one aspect, the surgical system further comprises a display. The control circuit is to transmit a signal to the display based on the value being below the seal threshold. The feedback comprises visual feedback on the display. The visual feedback is based on the signal.

In at least one aspect, the surgical system further comprises an audio feedback module. The control circuit is to transmit a signal to the audio feedback module based on the value being below the seal threshold. The feedback comprises audio feedback via the audio feedback module. The audio feedback is based on the signal.

In at least one aspect, the surgical system further comprises a haptic feedback module. The control circuit is to transmit a signal to a haptic feedback module based on the value being below the seal threshold. The feedback comprises haptic feedback via the haptic feedback module. The haptic feedback is based on the signal.

In at least one aspect, the RF energy source comprises the control circuit.

In at least one aspect, the surgical system further comprises a processing unit comprising the control circuit.

### BRIEF DESCRIPTION OF THE DRAWINGS

Various features of the embodiments described herein, together with advantages thereof, may be understood in accordance with the following description taken in conjunction with the accompanying drawings as follows:

FIG. 1 is a surgical hub paired with a visualization system, a robotic system, and an intelligent instrument, according to at least one aspect of the present disclosure.

FIG. 2 illustrates a control circuit to control aspects of the surgical instrument or tool, according to at least one aspect of the present disclosure.

FIG. 3 illustrates a combinational logic circuit to control aspects of the surgical instrument or tool, according to at least one aspect of the present disclosure.

FIG. 4 illustrates a sequential logic circuit to control aspects of the surgical instrument or tool, according to at least one aspect of the present disclosure.

FIG. 5 is a schematic diagram of a surgical instrument to control various functions, according to at least one aspect of the present disclosure.

FIG. 6 is a diagram of various modules and other components that are combinable to customize modular energy systems, according to at least one aspect of the present disclosure.

FIG. 7 is a video rating system for surgical recordings, in accordance with at least one aspect of the present disclosure.

FIG. 8 is a surgical view from a camera of a surgical instrument with jaws open, in accordance with at least one aspect of the present disclosure.

FIG. 9 is a surgical view from a camera of a surgical instrument with jaws clamped on tissue, in accordance with at least one aspect of the present disclosure.

FIG. 10 is a surgical view from a camera of a surgical instrument with jaws clamped after cutting and sealing tissue, in accordance with at least one aspect of the present disclosure.

FIG. 11 is a diagram of a control process to monitor end effector movement, in accordance with at least one aspect of the present disclosure.

FIG. 12 is a graphical representation of movement of a powered stapler vs a non-powered stapler, in accordance with at least one aspect of the present disclosure.

FIG. 13 is a surgical view from a camera of a surgical instrument having fiducial marks, in accordance with at least one aspect of the present disclosure.

FIG. 14 is a surgical view from a camera where a tissue tension alert is superimposed on the view, in accordance with at least one aspect of the present disclosure.

FIG. 15 is a diagram of a control process to calculate tissue tension, in accordance with at least one aspect of the present disclosure.

FIG. 16 is a representation of axial tissue tension, in accordance with at least one aspect of the present disclosure.

FIG. 17 is a representation of radial tissue tension, in accordance with at least one aspect of the present disclosure.

FIG. 18 illustrates a flow chart, according to at least one aspect of the present disclosure.

FIGS. 19A-C illustrates video labels, according to at least one aspect of the present disclosure.

FIG. 20 illustrates a flowchart of multi-task learning during model training and validation, according to at least one aspect of the present disclosure.

FIG. 21 illustrates a flowchart of multi-task leaning during verification, validation, and clinical use, according to at least one aspect of the present disclosure.

FIG. 22 illustrates a composite load curve look-up table, according to at least one aspect of the present disclosure.

FIGS. 23-25 illustrate wavelet signal processing, according to at least one aspect of the present disclosure.

FIG. 26 illustrates electrical parameters split into ‘x’ time length segments, according to at least one aspect of the present disclosure.

FIGS. 27-29 illustrate an implementation of Synthetic Minority Oversampling Technique (“SMOTE”), according to at least one aspect of the present disclosure.

FIG. 30 illustrates PCA identifying relevant features, according to at least one aspect of the present disclosure.

FIG. 31 illustrates an xgboost that allows model tuning back on multiple parameters, according to at least one aspect of the present disclosure.

FIGS. 32A and 32B illustrate model architecture for a CNN model, according to at least one aspect of the present disclosure.

FIG. 33 illustrates a confusion matrix, according to at least one aspect of the present disclosure.

FIG. 34 illustrates a ROC curve, according to at least one aspect of the present disclosure.

FIG. 35 illustrates a confusion matrix, according to at least one aspect of the present disclosure.

FIG. 36A-36D illustrate four confusion matrices, according to at least one aspect of the present disclosure.

FIG. 37 illustrates visual feedback on a display indicating a good quality seal, according to at least one aspect of the present disclosure.

FIG. 38 illustrates visual feedback on a display indicating a poor quality seal, according to at least one aspect of the present disclosure.

FIG. 39 illustrates a first embodiment of a seal quality prediction system, according to at least one aspect of the present disclosure.

FIG. 40 illustrates a second embodiment of a seal quality prediction system, according to at least one aspect of the present disclosure.

Corresponding reference characters indicate corresponding parts throughout the several views. The exemplifications set out herein illustrate various embodiments of the invention, in one form, and such exemplifications are not to be construed as limiting the scope of the invention in any manner.

### DETAILED DESCRIPTION

Numerous specific details are set forth to provide a thorough understanding of the overall structure, function, manufacture, and use of the embodiments as described in the specification and illustrated in the accompanying drawings. Well-known operations, components, and elements have not been described in detail so as not to obscure the embodiments described in the specification. The reader will understand that the embodiments described and illustrated herein are non-limiting examples, and thus it can be appreciated that the specific structural and functional details disclosed herein may be representative and illustrative. Variations and changes thereto may be made without departing from the scope of the claims.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a surgical system, device, or apparatus that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Various exemplary devices and methods are provided for performing laparoscopic and minimally invasive surgical procedures. However, the reader will readily appreciate that the various methods and devices disclosed herein can be used in numerous surgical procedures and applications including, for example, in connection with open surgical procedures. As the present Detailed Description proceeds, the reader will further appreciate that the various instruments disclosed herein can be inserted into a body in any way, such as through a natural orifice, through an incision or puncture hole formed in tissue, etc. The working portions or end effector portions of the instruments can be inserted directly into a patient's body or can be inserted through an access device that has a working channel through which the end effector and elongate shaft of a surgical instrument can be advanced.

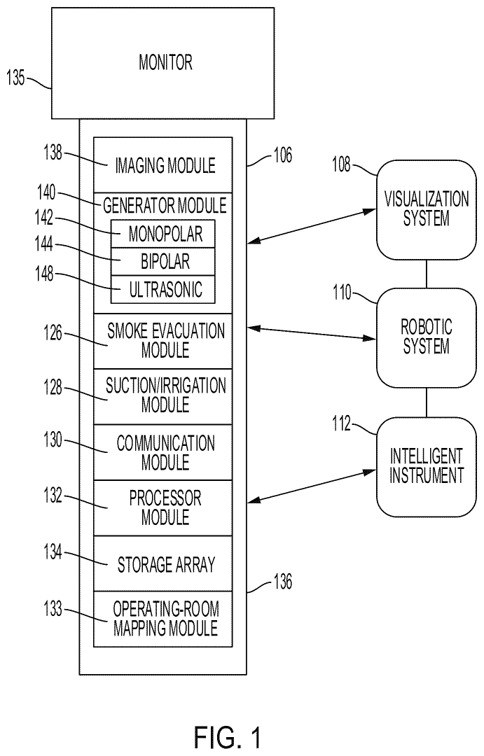

Referring to FIG. 1, a hub 106 is depicted in communication with a visualization system 108, a robotic system 110, and a handheld intelligent surgical instrument 112. In some aspects, the visualization system 108 may be a separable piece of equipment. In alternative aspects, the visualization system 108 could be contained within the hub 106 as a functional module. The hub 106 includes a hub display 135, an imaging module 138, a generator module 140, a communication module 130, a processor module 132, a storage array 134, and an operating room mapping module 133. In certain aspects, as illustrated in FIG. 1, the hub 106 further includes a smoke evacuation module 126, a suction/irrigation module 128, and/or an insufflation module 129. In certain aspects, any of the modules in the hub 106 may be combined with each other into a single module.

During a surgical procedure, energy application to tissue, for sealing and/or cutting, is generally associated with smoke evacuation, suction of excess fluid, and/or irrigation of the tissue. Fluid, power, and/or data lines from different sources are often entangled during the surgical procedure. Valuable time can be lost addressing this issue during a surgical procedure. Detangling the lines may necessitate disconnecting the lines from their respective modules, which may require resetting the modules. The hub modular enclosure 136 offers a unified environment for managing the power, data, and fluid lines, which reduces the frequency of entanglement between such lines.

Aspects of the present disclosure present a surgical hub for use in a surgical procedure that involves energy application to tissue at a surgical site. The surgical hub includes a hub enclosure and a combo generator module slidably receivable in a docking station of the hub enclosure. The docking station includes data and power contacts. The combo generator module includes one or more of an ultrasonic energy generator component, a bipolar RF energy generator component, and a monopolar RF energy generator component that are housed in a single unit. In one aspect, the combo generator module also includes a smoke evacuation component, at least one energy delivery cable for connecting the combo generator module to a surgical instrument, at least one smoke evacuation component to evacuate smoke, fluid, and/or particulates generated by the application of therapeutic energy to the tissue, and a fluid line extending from the remote surgical site to the smoke evacuation component.

In one aspect, the fluid line is a first fluid line and a second fluid line extends from the remote surgical site to a suction and irrigation module slidably received in the hub enclosure. In one aspect, the hub enclosure comprises a fluid interface.

Certain surgical procedures may require the application of more than one energy type to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 136 is to accommodate different generators, and facilitate an interactive communication therebetween. One of the advantages of the hub modular enclosure 136 is enabling the quick removal and/or replacement of various modules.

Aspects of the present disclosure present a modular surgical enclosure for use in a surgical procedure that involves energy application to tissue. The modular surgical enclosure includes a first energy-generator module, to generate a first energy for application to the tissue, and a first docking station comprising a first docking port that includes first data and power contacts. In one aspect, the first energy-generator module is slidably movable into an electrical engagement with the power and data contacts and wherein the first energy-generator module is slidably movable out of the electrical engagement with the first power and data contacts. In an alternative aspect, the first energy-generator module is stackably movable into an electrical engagement with the power and data contacts and wherein the first energy-generator module is stackably movable out of the electrical engagement with the first power and data contacts.

Further to the above, the modular surgical enclosure also includes a second energy-generator module to generate a second energy, either the same or different than the first energy, for application to the tissue, and a second docking station comprising a second docking port that includes second data and power contacts. In one aspect, the second energy-generator module is slidably movable into an electrical engagement with the power and data contacts, and wherein the second energy-generator module is slidably movable out of the electrical engagement with the second power and data contacts. In an alternative aspect, the second energy-generator module is stackably movable into an electrical engagement with the power and data contacts, and wherein the second energy-generator module is stackably movable out of the electrical engagement with the second power and data contacts.

In addition, the modular surgical enclosure also includes a communication bus between the first docking port and the second docking port, to facilitate communication between the first energy-generator module and the second energy-generator module.

Referring to FIG. 1, aspects of the present disclosure are presented for a hub modular enclosure 136 that allows the modular integration of a generator module 140, a smoke evacuation module 126, a suction/irrigation module 128, and an insufflation module 129. The hub modular enclosure 136 further facilitates interactive communication between the modules 140, 126, 128, 129. The generator module 140 can be a generator module with integrated monopolar, bipolar, and ultrasonic components supported in a single housing unit slidably insertable into the hub modular enclosure 136. The generator module 140 can be to connect to a monopolar device 142, a bipolar device 144, and an ultrasonic device 148. Alternatively, the generator module 140 may comprise a series of monopolar, bipolar, and/or ultrasonic generator modules that interact through the hub modular enclosure 136. The hub modular enclosure 136 can be to facilitate the insertion of multiple generators and interactive communication between the generators docked into the hub modular enclosure 136 so that the generators would act as a single generator.

In one aspect, the hub modular enclosure 136 comprises a modular power and communication backplane 149 with external and wireless communication headers to enable the removable attachment of the modules 140, 126, 128, 129 and interactive communication therebetween.

Additional information regarding the hub can be found in U.S. Patent Application Publication No. 2019/0201136 and U.S. Patent Application Publication No. 2020/0078106, which are hereby incorporated by reference in their entireties herein.

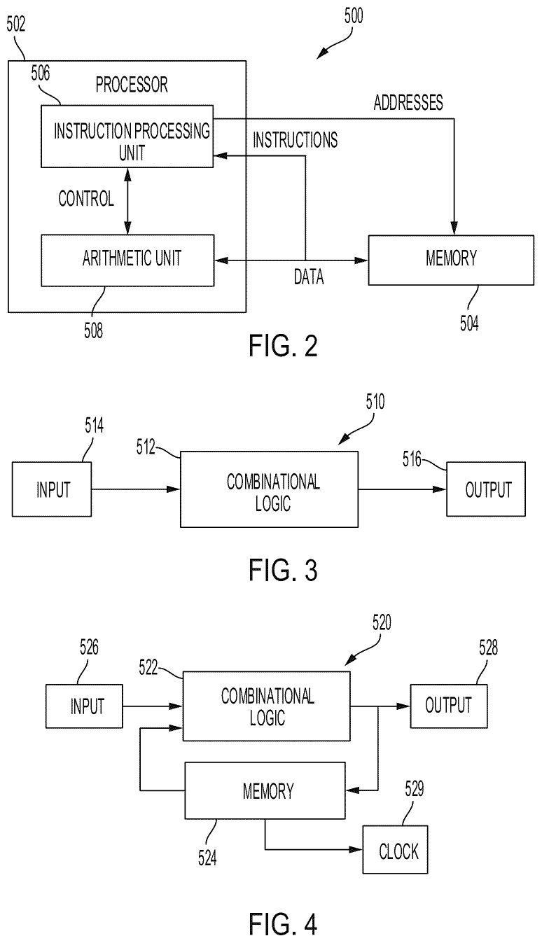

FIG. 2 illustrates a control circuit 500 to control aspects of the surgical instrument or tool according to one aspect of this disclosure. The control circuit 500 can be to implement various processes described herein. The control circuit 500 may comprise a microcontroller comprising one or more processors 502 (e.g., microprocessor, microcontroller) coupled to at least one memory circuit 504. The memory circuit 504 stores machine-executable instructions that, when executed by the processor 502, cause the processor 502 to execute machine instructions to implement various processes described herein. The processor 502 may be any one of a number of single-core or multicore processors known in the art. The memory circuit 504 may comprise volatile and non-volatile storage media. The processor 502 may include an instruction processing unit 506 and an arithmetic unit 508. The instruction processing unit may be to receive instructions from the memory circuit 504 of this disclosure.

FIG. 3 illustrates a combinational logic circuit 510 to control aspects of the surgical instrument or tool according to one aspect of this disclosure. The combinational logic circuit 510 can be to implement various processes described herein. The combinational logic circuit 510 may comprise a finite state machine comprising a combinational logic 512 to receive data associated with the surgical instrument or tool at an input 514, process the data by the combinational logic 512, and provide an output 516.

FIG. 4 illustrates a sequential logic circuit 520 to control aspects of the surgical instrument or tool according to one aspect of this disclosure. The sequential logic circuit 520 or the combinational logic 522 can be to implement various processes described herein. The sequential logic circuit 520 may comprise a finite state machine. The sequential logic circuit 520 may comprise a combinational logic 522, at least one memory circuit 524, and a clock 529, for example. The at least one memory circuit 524 can store a current state of the finite state machine. In certain instances, the sequential logic circuit 520 may be synchronous or asynchronous. The combinational logic 522 is to receive data associated with the surgical instrument or tool from an input 526, process the data by the combinational logic 522, and provide an output 528. In other aspects, the circuit may comprise a combination of a processor (e.g., processor 502, FIG. 2) and a finite state machine to implement various processes herein. In other aspects, the finite state machine may comprise a combination of a combinational logic circuit (e.g., combinational logic circuit 510, FIG. 3) and the sequential logic circuit 520.

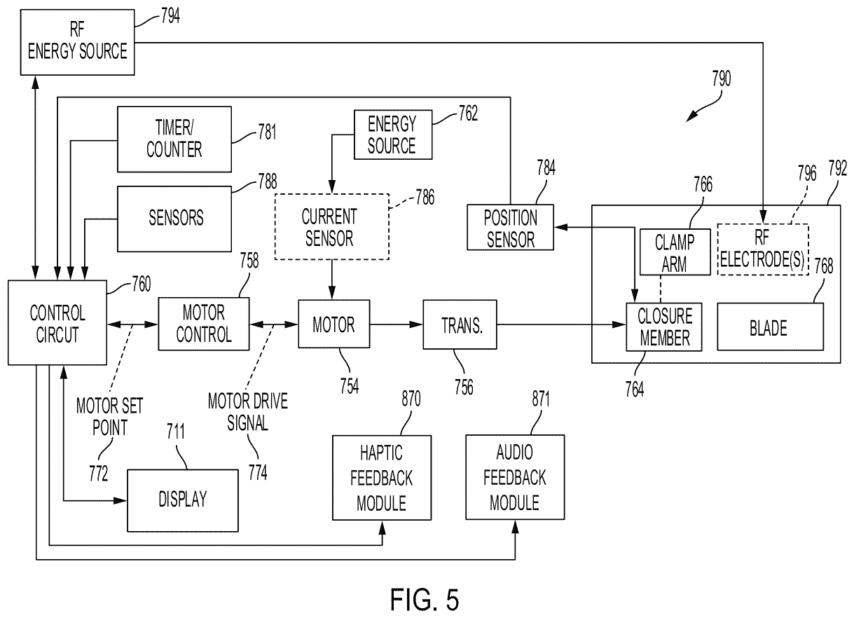

FIG. 5 is a schematic diagram of a surgical instrument 790 to control various functions according to one aspect of this disclosure. In one aspect, the surgical instrument 790 is programmed to control distal translation of a displacement member such as the closure member 764. The surgical instrument 790 comprises an end effector 792 that may comprise a clamp arm 766, a closure member 764, and a blade 768 which may be interchanged with or work in conjunction with one or more RF electrodes 796 (shown in dashed line). In various embodiments, the blade can comprise an I-beam, such as those described elsewhere herein. In various embodiments, the blade can comprise an ultrasonic blade that is coupled to an ultrasonic transducer driven by an ultrasonic generator.

In one aspect, sensors 788 may be implemented as a limit switch, electromechanical device, solid-state switches, Hall-effect devices, MR devices, GMR devices, magnetometers, among others. In other implementations, the sensors 638 may be solid-state switches that operate under the influence of light, such as optical sensors, IR sensors, ultraviolet sensors, among others. Still, the switches may be solid-state devices such as transistors (e.g., FET, junction FET, MOSFET, bipolar, and the like). In other implementations, the sensors 788 may include electrical conductorless switches, ultrasonic switches, accelerometers, and inertial sensors, among others.

In one aspect, the position sensor 784 may be implemented as an absolute positioning system comprising a magnetic rotary absolute positioning system implemented as an AS5055EQFT single-chip magnetic rotary position sensor available from Austria Microsystems, AG. The position sensor 784 may interface with the control circuit 760 to provide an absolute positioning system. The position may include multiple Hall-effect elements located above a magnet and coupled to a CORDIC processor, also known as the digit-by-digit method and Volder's algorithm, that is provided to implement a simple and efficient algorithm to calculate hyperbolic and trigonometric functions that require only addition, subtraction, bitshift, and table lookup operations.

In some examples, the position sensor 784 may be omitted. Where the motor 754 is a stepper motor, the control circuit 760 may track the position of the closure member 764 by aggregating the number and direction of steps that the motor has been instructed to execute. The position sensor 784 may be located in the end effector 792 or at any other portion of the instrument.

The control circuit 760 may be in communication with one or more sensors 788. The sensors 788 may be positioned on the end effector 792 and adapted to operate with the surgical instrument 790 to measure the various derived parameters such as gap distance versus time, tissue compression versus time, and anvil strain versus time. The sensors 788 may comprise a magnetic sensor, a magnetic field sensor, a strain gauge, a pressure sensor, a force sensor, an inductive sensor such as an eddy current sensor, a resistive sensor, a capacitive sensor, an optical sensor, and/or any other suitable sensor for measuring one or more parameters of the end effector 792. The sensors 788 may include one or more sensors.

An RF energy source 794 is coupled to the end effector 792 and is applied to the RF electrode 796 when the RF electrode 796 is provided in the end effector 792 in place of the blade 768 or to work in conjunction with the blade 768. For example, the blade is made of electrically conductive metal and may be employed as the return path for electrosurgical RF current. The control circuit 760 controls the delivery of the RF energy to the RF electrode 796.

The control circuit 760 may be in communication a haptic feedback module 870. In some embodiments, the haptic feedback module 870 is positioned in a handpiece, such as within the hand-pieces 1205, 1207, 1209 of the surgical instruments 1204, 1206, 1208, described in more detail below. In some embodiments, the haptic feedback module 870 is positioned within an input interface that a user interacts with to control the various surgical instruments described elsewhere herein. The control circuit 760 can transmit a control signal to the haptic feedback module 870 to cause the haptic feedback module 870 to actuate and provide haptic feedback, as described in more detail elsewhere herein.

The control circuit 760 may be in communication an audio feedback module 871. The control circuit 760 can transmit a control signal to the audio feedback module 871 to cause the audio feedback module 871 to actuate and provide audio feedback to a user, as described in more detail elsewhere herein.

Additional details are disclosed in U.S. patent application Ser. No. 15/636,096, titled SURGICAL SYSTEM COUPLABLE WITH STAPLE CARTRIDGE AND RADIO FREQUENCY CARTRIDGE, AND METHOD OF USING SAME, filed Jun. 28, 2017, which is herein incorporated by reference in its entirety.

### Generator Hardware

As used throughout this description, the term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some aspects they might not. The communication module may implement any of a number of wireless or wired communication standards or protocols, including but not limited to Wi-Fi (IEEE 802.11 family), WiMAX (IEEE 802.16 family), IEEE 802.20, long term evolution (LTE), Ev-DO, HSPA+, HSDPA+, HSUPA+, EDGE, GSM, GPRS, CDMA, TDMA, DECT, Bluetooth, Ethernet derivatives thereof, as well as any other wireless and wired protocols that are designated as 3G, 4G, 5G, and beyond. The computing module may include a plurality of communication modules. For instance, a first communication module may be dedicated to shorter range wireless communications such as Wi-Fi and Bluetooth and a second communication module may be dedicated to longer range wireless communications such as GPS, EDGE, GPRS, CDMA, WiMAX, LTE, Ev-DO, and others.

As used herein a processor or processing unit is an electronic circuit which performs operations on some external data source, usually memory or some other data stream. The term is used herein to refer to the central processor (central processing unit) in a system or computer systems (especially systems on a chip (SoCs)) that combine a number of specialized “processors.”

As used herein, a system on a chip or system on chip (SoC or SOC) is an integrated circuit (also known as an “IC” or “chip”) that integrates all components of a computer or other electronic systems. It may contain digital, analog, mixed-signal, and often radio-frequency functions—all on a single substrate. A SoC integrates a microcontroller (or microprocessor) with advanced peripherals like graphics processing unit (GPU), Wi-Fi module, or coprocessor. A SoC may or may not contain built-in memory.

As used herein, a microcontroller or controller is a system that integrates a microprocessor with peripheral circuits and memory. A microcontroller (or MCU for microcontroller unit) may be implemented as a small computer on a single integrated circuit. It may be similar to a SoC; a SoC may include a microcontroller as one of its components. A microcontroller may contain one or more core processing units (CPUs) along with memory and programmable input/output peripherals. Program memory in the form of Ferroelectric RAM, NOR flash or OTP ROM is also often included on chip, as well as a small amount of RAM. Microcontrollers may be employed for embedded applications, in contrast to the microprocessors used in personal computers or other general purpose applications consisting of various discrete chips.

As used herein, the term controller or microcontroller may be a stand-alone IC or chip device that interfaces with a peripheral device. This may be a link between two parts of a computer or a controller on an external device that manages the operation of (and connection with) that device.

Any of the processors or microcontrollers described herein, may be implemented by any single core or multicore processor such as those known under the trade name ARM Cortex by Texas Instruments. In one aspect, the processor may be an LM4F230H5QR ARM Cortex-M4F Processor Core, available from Texas Instruments, for example, comprising on-chip memory of 256 KB single-cycle flash memory, or other non-volatile memory, up to 40 MHz, a prefetch buffer to improve performance above 40 MHz, a 32 KB single-cycle serial random access memory (SRAM), internal read-only memory (ROM) loaded with StellarisWare® software, 2 KB electrically erasable programmable read-only memory (EEPROM), one or more pulse width modulation (PWM) modules, one or more quadrature encoder inputs (QEI) analog, one or more 12-bit Analog-to-Digital Converters (ADC) with 12 analog input channels, details of which are available for the product datasheet.

In one aspect, the processor may comprise a safety controller comprising two controller-based families such as TMS570 and RM4x known under the trade name Hercules ARM Cortex R4, also by Texas Instruments. The safety controller may be configured specifically for IEC 61508 and ISO 26262 safety critical applications, among others, to provide advanced integrated safety features while delivering scalable performance, connectivity, and memory options.

Modular devices include the modules (as described in connection with FIG. 1, for example) that are receivable within a surgical hub and the surgical devices or instruments that can be connected to the various modules in order to connect or pair with the corresponding surgical hub. The modular devices include, for example, intelligent surgical instruments, medical imaging devices, suction/irrigation devices, smoke evacuators, energy generators, ventilators, insufflators, and displays. The modular devices described herein can be controlled by control algorithms. The control algorithms can be executed on the modular device itself, on the surgical hub to which the particular modular device is paired, or on both the modular device and the surgical hub (e.g., via a distributed computing architecture). In some exemplifications, the modular devices' control algorithms control the devices based on data sensed by the modular device itself (i.e., by sensors in, on, or connected to the modular device). This data can be related to the patient being operated on (e.g., tissue properties or insufflation pressure) or the modular device itself (e.g., the rate at which a knife is being advanced, motor current, or energy levels). For example, a control algorithm for a surgical stapling and cutting instrument can control the rate at which the instrument's motor drives its knife through tissue according to resistance encountered by the knife as it advances.

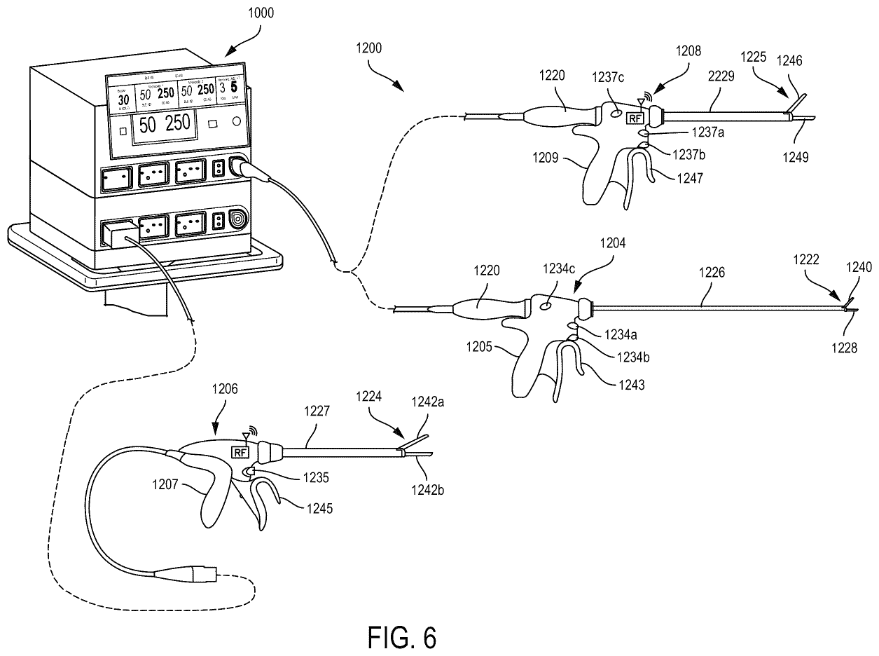

FIG. 6 illustrates one form of a surgical system 1200 comprising a modular energy system 1000 and various surgical instruments 1204, 1206, 1208 usable therewith, where the surgical instrument 1204 is an ultrasonic surgical instrument, the surgical instrument 1206 is an RF electrosurgical instrument, and the multifunction surgical instrument 1208 is a combination ultrasonic/RF electrosurgical instrument. The modular energy system 1000 is configurable for use with a variety of surgical instruments. According to various forms, the modular energy system 1000 may be configurable for use with different surgical instruments of different types including, for example, ultrasonic surgical instruments 1204, RF electrosurgical instruments 1206, and multifunction surgical instruments 1208 that integrate RF and ultrasonic energies delivered individually or simultaneously from the modular energy system 1000. Although in the form of FIG. 6 the modular energy system 1000 is shown separate from the surgical instruments 1204, 1206, 1208 in one form, the modular energy system 1000 may be formed integrally with any of the surgical instruments 1204, 1206, 1208 to form a unitary surgical system. The modular energy system 1000 may be configured for wired or wireless communication.

The modular energy system 1000 is to drive multiple surgical instruments 1204, 1206, 1208. The first surgical instrument is an ultrasonic surgical instrument 1204 and comprises a hand-piece 1205 (HP), an ultrasonic transducer 1220, a shaft 1226, and an end effector 1222. The end effector 1222 comprises an ultrasonic blade 1228 acoustically coupled to the ultrasonic transducer 1220 and a clamp arm 1240. The hand-piece 1205 comprises a trigger 1243 to operate the clamp arm 1240 and a combination of the toggle buttons 1234a, 1234b, 1234c to energize and drive the ultrasonic blade 1228 or other function. The toggle buttons 1234a, 1234b, 1234c can be to energize the ultrasonic transducer 1220 with the modular energy system 1000.

The modular energy system 1000 also is to drive a second surgical instrument 1206. The second surgical instrument 1206 is an RF electrosurgical instrument and comprises a hand-piece 1207 (HP), a shaft 1227, and an end effector 1224. The end effector 1224 comprises electrodes in clamp arms 1242a, 1242b and return through an electrical conductor portion of the shaft 1227. The electrodes are coupled to and energized by a bipolar energy source within the modular energy system 1000. The hand-piece 1207 comprises a trigger 1245 to operate the clamp arms 1242a, 1242b and an energy button 1235 to actuate an energy switch to energize the electrodes in the end effector 1224.

The modular energy system 1000 also is to drive a multifunction surgical instrument 1208. The multifunction surgical instrument 1208 comprises a hand-piece 1209 (HP), a shaft 1229, and an end effector 1225. The end effector 1225 comprises an ultrasonic blade 1249 and a clamp arm 1246. The ultrasonic blade 1249 is acoustically coupled to the ultrasonic transducer 1220. The ultrasonic transducer 1220 may be separable from or integral to the hand-piece 1209. The hand-piece 1209 comprises a trigger 1247 to operate the clamp arm 1246 and a combination of the toggle buttons 1237a, 1237b, 1237c to energize and drive the ultrasonic blade 1249 or other function. The toggle buttons 1237a, 1237b, 1237c can be to energize the ultrasonic transducer 1220 with the modular energy system 1000 and energize the ultrasonic blade 1249 with a bipolar energy source also contained within the modular energy system 1000.

The modular energy system 1000 is configurable for use with a variety of surgical instruments. According to various forms, the modular energy system 1000 may be configurable for use with different surgical instruments of different types including, for example, the ultrasonic surgical instrument 1204, the RF electrosurgical instrument 1206, and the multifunction surgical instrument 1208 that integrates RF and ultrasonic energies delivered individually or simultaneously from the modular energy system 1000. Although in the form of FIG. 6 the modular energy system 1000 is shown separate from the surgical instruments 1204, 1206, 1208, in another form the modular energy system 1000 may be formed integrally with any one of the surgical instruments 1204, 1206, 1208 to form a unitary surgical system. Further aspects of generators for digitally generating electrical signal waveforms and surgical instruments are described in US patent publication US-2017-0086914-A1, which is herein incorporated by reference in its entirety.

Additional information regarding the modular energy system can be found in U.S. patent application Ser. No. 17/217,394, entitled “METHOD FOR MECHANICAL PACKAGING FOR MODULAR ENERGY SYSTEM”, filed Mar. 30, 2021, which is hereby incorporated by reference in its entirety herein.

### Tissue Tension

Having described a general implementation of a modular energy system 1000 and some surgical instruments that can couple to it, the disclosure now turns to the issue of tissue tension with surgical devices and the capability to calculate tissue tension. Tissue tension is an important parameter for the performance of both energy and endomechanical devices and can negatively affect sealing or stapling quality if used inappropriately. Stated another way, tissue tension affects all surgical devices that clamp on tissue such as staplers, clip appliers, ultrasonic devices, suturing devices, bipolar devices, mono polar devices, and graspers. Having poor control over the device can result in more tissue tension, which can damage the tissue that is being treated and negatively affect surgical outcomes. Currently, many surgical devices, such as handheld surgical devices, do not have the capability to determine tissue tension.

The present disclosure provides various solutions to calculate tissue tension and alert a user of the current tissue tension. This can allow a surgeon to maintain a low tissue tension helping them generate beneficial surgical outcomes. A control circuit of a surgical device can be coupled to sensors that provide it with information. The information can allow the control circuit to determine tissue tension and alert the user. The control circuit could calculate the tissue tension quantitatively or qualitatively. Some qualitative examples could be “Low”, “Medium”, “High”, “Lower”/“Falling”, or “Higher”/“Rising”. The user could be alerted to the tissue tension through visual or auditory feedback in real-time. In some instances, the user could also be provided feedback on the tissue tension post operatively.

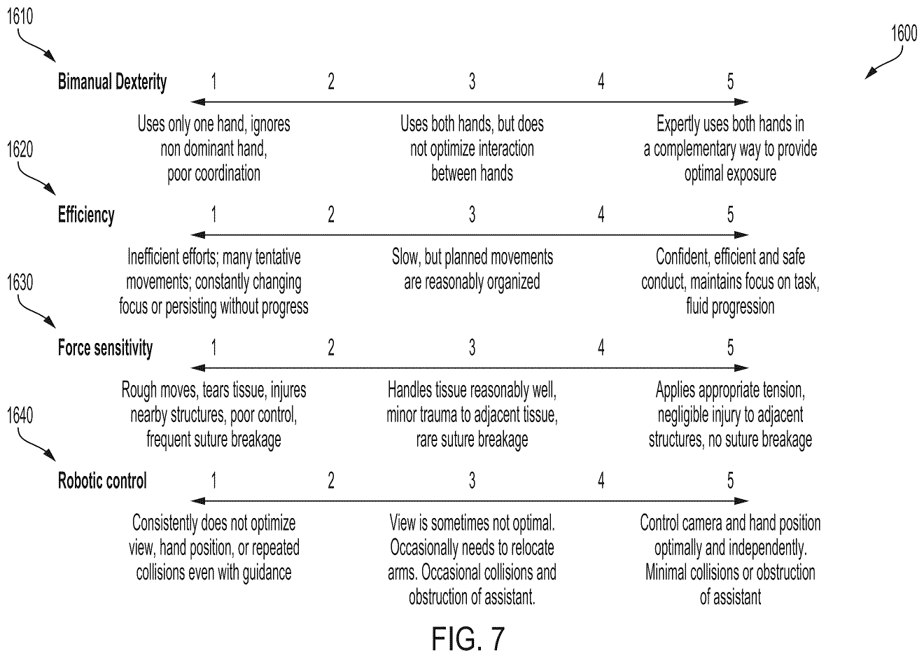

FIG. 7 illustrates a post-operative surgical video rating system that allows unbiased observers to use the rating system 1600 to rate how a surgeon performed in a surgical video recording. The rating system 1600 covers bimanual dexterity 1610, efficiency 1620, force sensitivity 1630, and robotic control 1640. A goal of the rating system 1600 is to provide a surgeon with unbiased feedback on their performance so that they can improve surgical outcomes. Providing the surgeon with real-time feedback on tissue tension during a surgery could help them improve their force sensitivity 1630 rating. Having high tissue tension can lead to among other things the tissue tearing and/or damaging nearby structures which lowers the surgeon's force sensitivity rating 1630. If the surgeon is suturing, a high tissue tension could lead to frequent suture breaks also lowering the force sensitivity rating 1630.

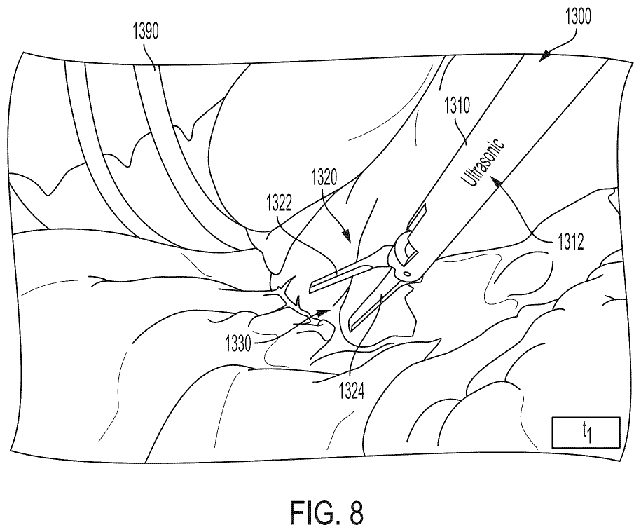

FIGS. 8-10 show the view 1390 provided to a surgeon as they perform a surgical procedure. The surgical device 1300 has a shaft 1310 that was inserted into a patient. The shaft 1310 ends at an end effector 1320. The end effector 1320 has a first jaw 1322 and a second jaw 1324. The first jaw 1322 is to rotate relative to the second jaw 1324 from an open configuration to a closed configuration. The shaft 1310 of the surgical device 1300 has a label 1312 on it detailing the type of surgical device. For example, in FIGS. 8-10, the surgical device is an ultrasonic surgical device with the label 1312 ultrasonic. For the surgical device 1300, the first jaw 1322 could be a clamp arm and the second jaw 1324 could be a blade. However, if the surgical device was a stapler, then the first jaw could be an anvil and the second jaw could house a staple cartridge. The surgical device 1300 can comprise various sensors that provide information to a control circuit that controls the surgical device 1300. For example, the control circuit that controls the surgical device could be hub 106, control circuit 500, or control circuit 760. In one instance, the control circuit could be a part of the surgical device. In an alternative instance, the control circuit could be separate and communicably coupled to the surgical device.

The surgical device 1300 can include a tissue sensor that is to detect tissue disposed between the first jaw 1322 and the second jaw 1324. In one instance, the tissue sensor could be a capacitive sensor that could detect tissue between the jaws 1322, 1324. In an alternative instance, the tissue sensor could detect the continuity between the two jaws 1322, 1324 and determine if there is tissue between the jaws 1322, 1324. For example, the surgical device could use a return electrode to calculate the continuity. In the instance of the surgical device being a bipolar electrosurgical device the continuity could be directly calculated. The tissue sensor allows the control circuit to determine when there is tissue between the jaws 1322, 1324.

The surgical device 1300 can include a jaw closure sensor that is to detect the end effector in a closed configuration. In one instance, the jaw closure sensor could be a jaw closure switch, where the switch is activated when the jaws 1322, 1324 are closed. In an alternative instance, the jaw closure sensor could be jaw closure mechanism position sensors, where the position sensors can be used to determine where the first jaw 1322 is in the closure process. For example, the positon sensors could be used to determine when the jaws 1322, 1324 are closed.

The surgical device 1300 can include a motion sensor to detect motion of the end effector 1320 or motion of the device 1300. For example, the motion sensor could detect the end effector motion including orientation, position, velocity, and acceleration. In various instances, the motion sensor could include accelerometers, gyros, inertial measurement units, etc. The motion sensor could also be a combination of multiple sensors that provide the motion of the end effector. The motion sensor or motion sensors could be mounted in or on the surgical device 1300.

The control circuit can also determine when the surgical device 1300 is in a surgeon's hand. In some instances, the control circuit can use the motion sensors to determine when the surgical device 1300 is in use. In an alternative instance, the control circuit can use video analysis from a camera viewing the surgical site to determine when the surgical device 1300 enters the field of view. For example, the video analysis could detect the label 1312 on the device 1300 when the device 1300 is in the field of view. As another example, the video analysis could detect the geometry of the device 1300 to detect when the device 1300 is in the field of view.

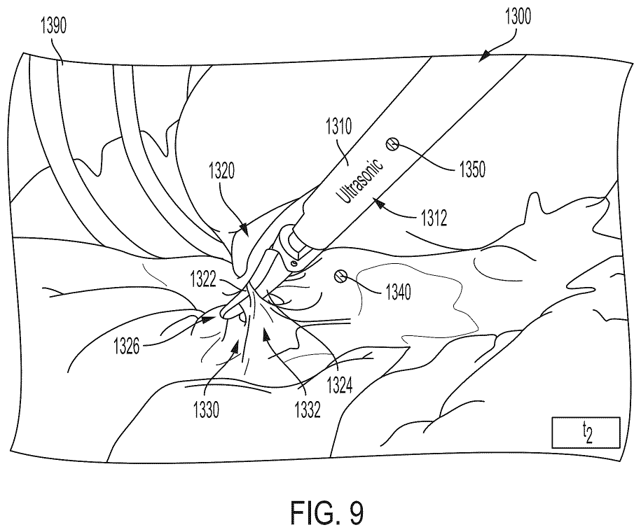

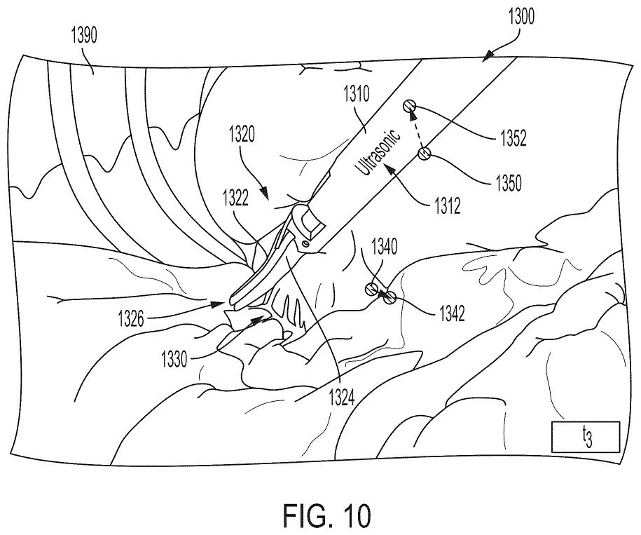

Referring to FIG. 8, the end effector 1320 of the surgical device 1300 is in an open configuration at a first time t₁ in the surgical procedure. In FIG. 9, the end effector 1320 has closed on tissue 1330 at a second time t₂ in the surgical procedure. The first time t₁ occurs before the second time t₂. The tip of the device 1300 is located at 1326. It can be seen in FIG. 9, that there is tension on the tissue from the tenting 1332 of the tissue. Point 1340 is a location on the tissue that can be monitored to see if the tissue moves after division of tissue. Point 1350 is a point on the surgical device 1300 that can be monitored to see how the device moves after division of the tissue. In FIG. 10, the tissue has been cut and sealed at a third time t₃ in the surgical procedure. The second time t₂ occurs before the third time t₃. It can be seen that there was an abrupt movement of the device 1300 following the division of the tissue. This is shown by point 1350 moving to point 1352. Additionally, the point on the tissue moved from 1340 to 1342 showing that the tissue also moved. These motions can be indicative of tissue tension while the tissue was clamped in device 1300. The motion of the device 1300 can be captured and/or measured and the event tagged for procedure analytics.

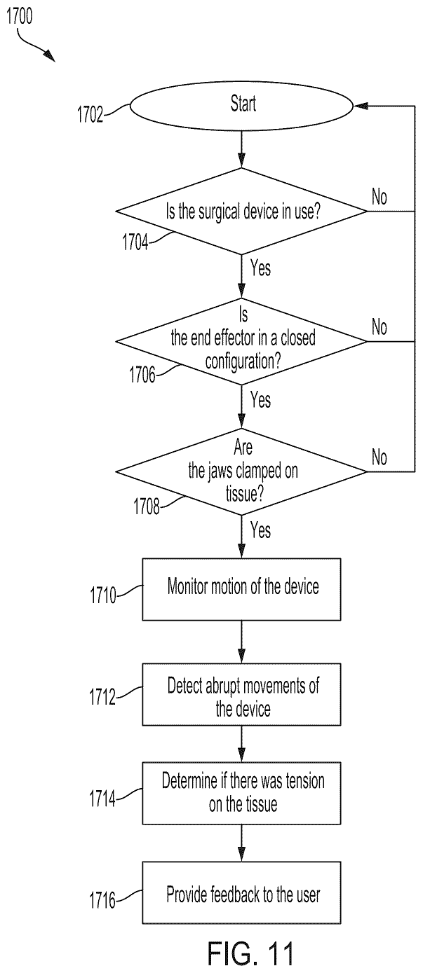

The control circuit can monitor device motion as an indicator of tissue tension in accordance with FIG. 11. In various instances, the control circuit can save computational resources by monitoring the device for tissue tension when the device is clamped on tissue. The control circuit process 1700 begins at 1702. The control circuit determines if the surgical device is in use 1704. If the surgical device is in use, then the control circuit proceeds to determine if the end effector is in a closed configuration 1706. If the surgical device is in a closed configuration, then the control circuit proceeds to determine if the jaws are clamped on tissue 1708. If the jaw is clamped on tissue, then the control circuit proceeds to monitor the motion of the device 1710. If the surgical device was not in use, the end effector was not in a closed configuration, or the jaws were not clamped on tissue, then the control circuit would start 1702 the process 1700 over again. This allows the control circuit to only monitor the motion of the device when the surgical device is clamped on tissue, such as in FIG. 9. The control circuit can monitor the movements of the device and look for any rough or abrupt movements that might indicate tissue tension 1712. For example, the control circuit can detect motion of the end effector outside of a predetermined range. The motion of the device can be scaled based on a known distance from the motion sensors to end of the device tip.

This monitoring of the motion includes monitoring the motion of the device at the end of activation when the tissue is separated or released. The end of activation can be detected through a change in the tissue sensor. It could also be detected through user de-activation or a change in the frequency for an ultrasonic device. Throughout monitoring the motion of the device, the control circuit can determine if there was tension on the tissue 1714. For example, an abrupt movement of the device following division of tissue can be indicative of tissue tension. The control circuit then proceeds to provide feedback to the user of tissue tension and/or abrupt or rough movements in a heads up display. For example, the feedback could be superimposed over the camera view 1390. The feedback could also be auditory. In either instance, the feedback can be provided to the surgeon in real-time allowing them to correct their movements to benefit the surgical outcome.

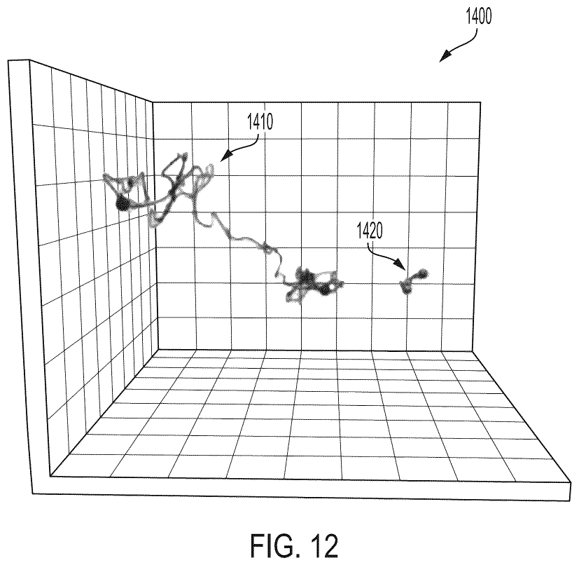

The feedback can also be provided to the surgeon post operatively. For example, the motion of the device throughout the surgical procedure can be provided to the user. Referring to FIG. 12, the surgeon could be provided diagram 1400 showing the movement of the tip of a surgical device during a surgical procedure. This can provide the surgeon with a view of how they moved the device and if there were abrupt or rough motions. Diagram 1400 shows movement of a powered surgical stapler 1420 preforming a surgical procedure and movement of a non-powered surgical stapler 1410 performing a similar surgical procedure. It can be seen that the powered surgical stapler provided 37% less movement due to the push button firing allowing surgeons to be more consistent between surgical procedures.

Additionally, abrupt or rough motions can be captured, measured, and the event tagged for procedure analytics. The rating system 1600 can also be used to provide a surgeon with an overall rating of their performance postoperatively. In various instances, the motion feedback could be broken into categories such as motion, motion while touching tissue, motion while clamped on tissue, and motion immediately following division or release of tissue. These categories could allow a surgeon to view the motion of the device during different aspects of the surgical procedure and see where they need to improve. It could also show if the surgeon continually has abrupt movements during a specific aspect of the surgical procedure. Overall, the movement feedback can be used to coach and enhance surgeon skills in real-time or in a postoperative report.

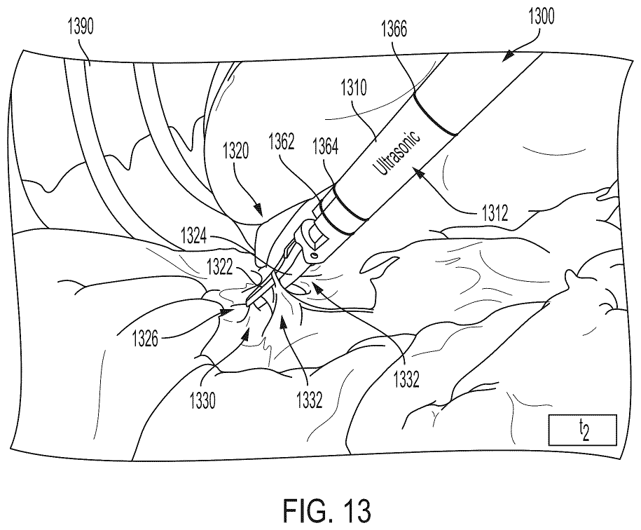

Referring to FIG. 13, the surgical device 1300 could include one or more fiducial marks 1362, 1364, and 1366. In various instances, the fiducial marks 1362, 1364, and 1366 could be rings that goes around the shaft 1310. In FIG. 13, each fiducial mark is a black ring around the shaft 1310, where the fiducial mark 1362 is distal to the fiducial mark 1364, which is distal to the fiducial mark 1366. In an alternative instance, the one or more fiducial marks could be any marked on the device 1300 that could be used by the control circuit. The fiducial marks 1362, 1364, and 1366 can be used to provide device identification. For example, the size and spacing of the fiducial marks 1362, 1364, and 1366 can be used by the control circuit to identify a surgical device or surgical device type. Additionally, the device type identity information could be known by way of blue tooth or direct wired communication to the OR system (e.g., energy generator). However, one or more fiducial marks on a device can be helpful for identifying a device when there are multiple surgical devices in the camera field of view. A label 1312 could also be used to identify multiple devices in the field of view. It would also be possible to use a datamatrix, QR codes, or etc. to identify multiple devices in the field of view.

The control circuit can use video data from the camera to calculate tissue tension during the surgical procedure. There can be an issue of computational burden when performing real-time video analysis. However, this burden can be mitigated by analyzing a small location within the video image and only performing the analysis over a specific time window based on events of interest. For example, an event of interest could be when the tissue is clamped in the jaws 1322, 1324 of the end effector 1320.

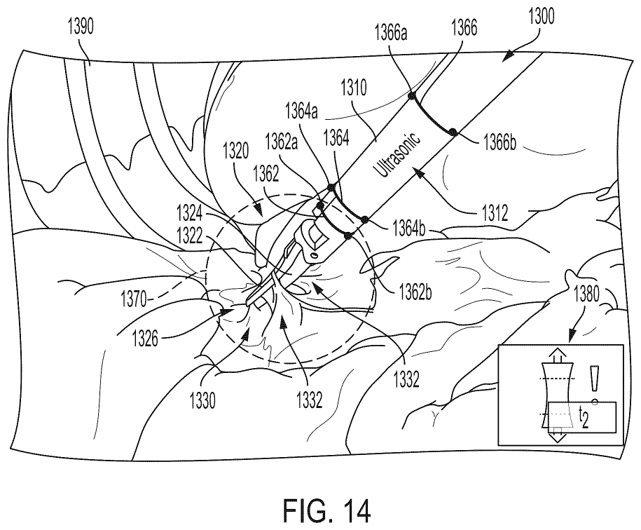

Referring to FIG. 14, the fiducial marks 1362, 1364, and 1364 can be used to define a vector (orientation and direction) in three-dimensional space that can be used to define the location of the tip 1326 of the device 1300. In various instances, the maximum extent of each ring, for example ends 1362a, 1362b of fiducial mark 1362, ends 1364a, 1364b of fiducial mark 1364, and ends 1366a, 1366b of fiducial mark 1366, can be used to generate a vector in three dimensional space. The location of the tip 1326 of the device 1300 in the video data can be calculated based on prior knowledge of the distance between the fiducial marks 1362, 1364, and 1366 and the tip 1326 of the device 1300. In an alternative instance, the control circuit could perform an analysis of the video data to locate the tip of the surgical instrument based on color and geometry of the surgical device. The location of the tip 1326 of the surgical device 1300 can be used to calculate a region of interest 1370 in the video image data. The region of interest 1370 can greatly reduce the computational burden by reducing the amount of space in the video that requires video analysis. Additionally, the computationally burden can be further reduced by only performing video analysis when the surgical device 1300 is clamped on tissue.

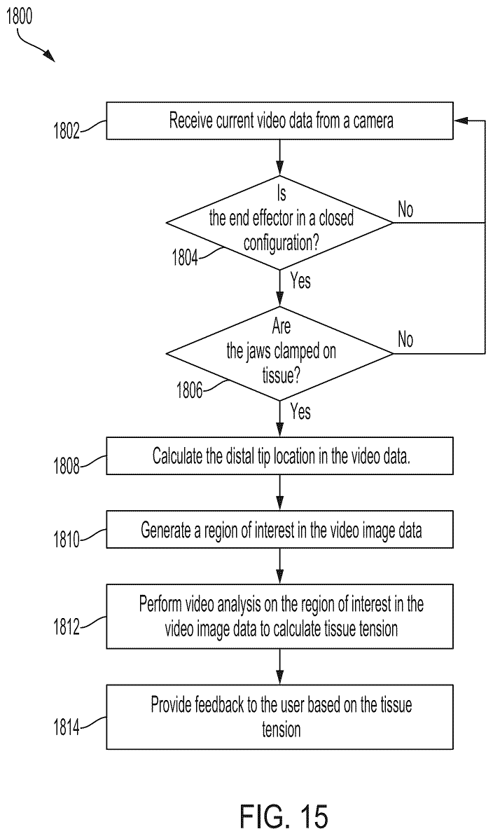

The control circuit can perform video analysis to calculate tissue tension in accordance with FIG. 15. In various instances, the control circuit can save computational resources by performing video analysis on a subset of the video data defined by a region of interest around the device tip. Additionally, the control circuit can choose to only perform the video analysis when the device is clamped on tissue, which further reduces the computational burden. The control circuit process 1800 begins by receiving current video data from a camera 1802 viewing the surgical site. The control circuit determines if the end effector is in a closed configuration 1804. If the end effector is in a closed configuration, then the control circuit proceeds to determine if the jaws are clamped on tissue 1806. If the jaw is clamped on tissue, then the control circuit proceeds to calculate the distal tip location in the video data 1808. If the end effector was not in a closed configuration or the jaws were not clamped on tissue, then the control circuit would start the process 1800 over again by receiving the next batch of video data from the camera 1802. This allows the control circuit to only perform the video analysis when the surgical device is clamped on tissue, such as in FIG. 14.

The distal tip location in the video data can be calculated by using one or more fiducial marks, such as fiducial marks 1362, 1364, and 1366 (FIG. 14), on the device. The one or more fiducial marks can be used to generate a vector in three dimensional space. The location of the device tip in the video data can be calculated based on prior knowledge of the distance between the one or more fiducial marks and the tip of the device. In an alternative instance, motion sensors could be used to calculate the tip of the device in three dimensional space.

Once the device tip location is known, the control circuit can generate a region of interest around the device tip location in the video data 1810. An example region of interest in video data is the region of interest 1370 shown in FIG. 14. This process reduces the amount of data that needs processed by the video analysis since the region of interest is only a subset of the video data. Once the region of interest is known, the control circuit can perform video analysis on the video image data to calculate tissue tension. Once the tissue tension is known, the control circuit can provide the tissue tension feedback to the user. For example, if tissue tension is detected then a color scaled tension alert could be superimposed on the video monitor, such as tissue tension alert 1380. The color scale could relate to a quantitative or qualitative rating system. For example, the color scale could relate to a qualitative scale of “low”, “medium” or “high” tissue tension. In an additional or alternative instance, the tissue tension alert could be auditory.

Referring to FIG. 14, the video analysis could calculate tissue tension by looking at the tissue 1330 in the region of interest 1370. The fiducial marks 1362, 1364, and 1366 provide video information with respect to the device tip 1326 location and orientation which is used to “see” tension. The video analysis could look for tenting 1332 in the tissue 1330 on one or both sides of the clamped end effector 1320. The tenting 1332 of the tissue 1330 can indicate the presence of tension and potentially the degree of tension. This information could then be provided to the surgeon through visual and/or auditory feedback.

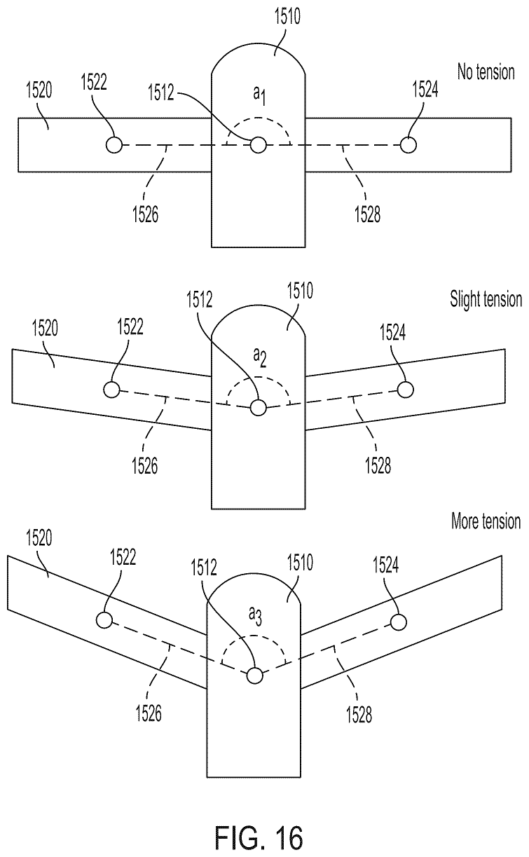

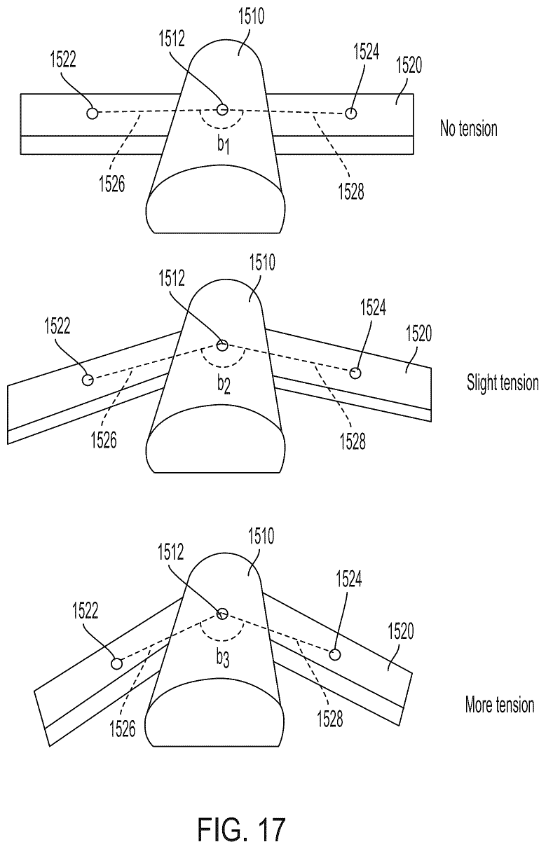

FIGS. 16 and 17 show two diagrams of tissue tension. FIG. 16 shows axial tissue tension and FIG. 17 shows radial tissue tension. The video analysis could use at least one fiducial marker, such as fiducial mark 1362, 1364, or 1366, added to the device to establish at least two points 1522, 1524 on the grasped tissue adjacent to the marker at some set relative distance. Using these points 1522, 1524, the system can establish two lines 1526, 1528, one line 1526 extending towards the grasped tissue on the left side of the end effector and one line 1528 extending towards the grasped tissue on the right side. The two lines intersect at point 1512 and the angle between the two lines can be used to assess the degree to which tissue is being pulled by the device either axially or radially. FIG. 16 shows axial tension, where the angle a₁ shows no tension, angle a₂ shows slight tension, and angle a₃ shows more tension. FIG. 17 shows radial tension, where the angle b₁ shows no tension, angle b₂ shows slight tension, and angle b₃ shows more tension. As more tension is applied to the tissue, the degree to which the angles “a” and “b” changes increases. For example, the change of angle a₁ to angle a₂ is less than the change of angle a₁ to a₃. Similarly, the change of angle b₁ to angle b₂ is less than the change of angle b₁ to b₃.

Tissue tension information can be translated to the device user via a visual (e.g. a low-medium-high scale) or an auditory (e.g. an alert tone is played if too much tension is applied) indicator. Relative changes over a range of video images can be used to see tension develop such as how the angles and lengths of edges change. This information can be provided to a surgeon in real-time for them to manage the tissue tension with the goal of minimizing tissue tension during the surgical procedure.

### Advanced Bipolar Seal Quality Predictions

Advanced Bipolar (“ABP”) tools are electrosurgical tools used in surgery for soft tissue dissection and vessel sealing and offer the primary benefits of reducing OR time and minimizing blood loss. These tools use radiofrequency current to create the heat needed for sealing, but the resultant temperatures are generally not sufficient to cause the tissue to cut. These ABP tools further utilize algorithms to ensure sealing completeness and a mechanically sharp knife, an ultrasonic blade, or any other suitable blade to cut tissue after sealing. Combining sealing and cutting into a single device enhances multifunctionality of the ABP tools, which increases their versatility, reduces the need for instrument exchanges into and out of the body, and enhances surgical efficiency, ease of use, and workflow.

Although the dissection and sealing leads to a desirable outcome most of the time, some of the activations of the ABP tool can still lead to minor or major intra-operative bleeding. Surgeons could use information about the quality of the seal to inform subsequent surgical maneuvers that would help prevent or mitigate bleeding, thus reducing patient blood loss and improving surgical efficiency.

The present disclosure provides a solution that can predict, in real time, poor seal quality with high likelihood of bleeding before it occurs. These predictions can occur during or after energy activation utilizing electrical and other measurements throughout the activation, as well as additional device data. Upon detection of poor seal quality, the system can issue a warning (auditory, visual or haptic, or combinations thereof) that allows the surgeon to decide what appropriate next steps should be taken to avoid bleeding, such as, for example, not mechanically cutting the tissue with the blade. The surgeon may also choose to reapply energy before transecting or perform other actions to prevent bleeding.

The seal quality prediction is based on an inference model that predicts, in near real-time, the likelihood of bleeding based on the data from the generator. The inference module is based on a pattern recognition model that is trained on annotated real-world data from surgeries on humans. The module can be deployed on digital hardware in the operating room with direct access to the device data.

In some embodiments, the system utilizes time-series electrical data throughout the activation of the instrument (current, voltage, impedance, power, sealing cycle phase, total energy, etc.) as well as device manufacturing data (clamp force, multi-point jaw gap measurements, device electrical impedance, etc.) as a basis for making predictions about seal quality. Information about the seal quality is the conveyed to the surgeon, who can then choose appropriate response. The surgical workflow is described in the “Concept” section, below.

To develop the algorithm, data is needed, which is described in the “Data Collection and Annotation” section, below. This data is preprocessed in real time into features through a number of different mathematical transformations and scaling operations, described in the “Feature Generation” section, below. Preprocessing may result in hundreds or thousands of features that are fed into the machine learning models, which takes the inputs and generates a numeric prediction, such as a variable scale from 0 to 1, as an example. In one embodiment, applying a threshold to this output, such as >0.5, as an example, this output can be transformed into a binary prediction for communication to the user. The machine learning models are described in the “Machine Learning (“ML”) Models” section, below.

In some embodiments, the threshold can be chosen for a particular model to appropriately balance the sensitivity and specificity of the prediction. To optimize the efficacy of the solution in the context of surgical workflow, it is important to balance out false positives (i.e. false alarms leading to alarm fatigue) as well as false negatives (i.e. missed bleeding events leading to bleeding). This is described in the “Metrics” section, below. Lastly, the information must be communicated to the surgeon. The electrosurgical system may provide its own interface, or work in conjunction with another system. This is described in the “Integration and Interface” section, below.

### Concept

As referenced above, in some embodiments, the system utilizes time-series electrical data throughout the activation of the instrument (current, voltage, impedance, power, sealing cycle phase, total energy, etc.) as well as device manufacturing data (clamp force, multi-point jaw gap measurements, device electrical impedance, etc.) as a basis for making predictions about seal quality. Information about the seal quality is the conveyed to the surgeon, who can then choose appropriate response.

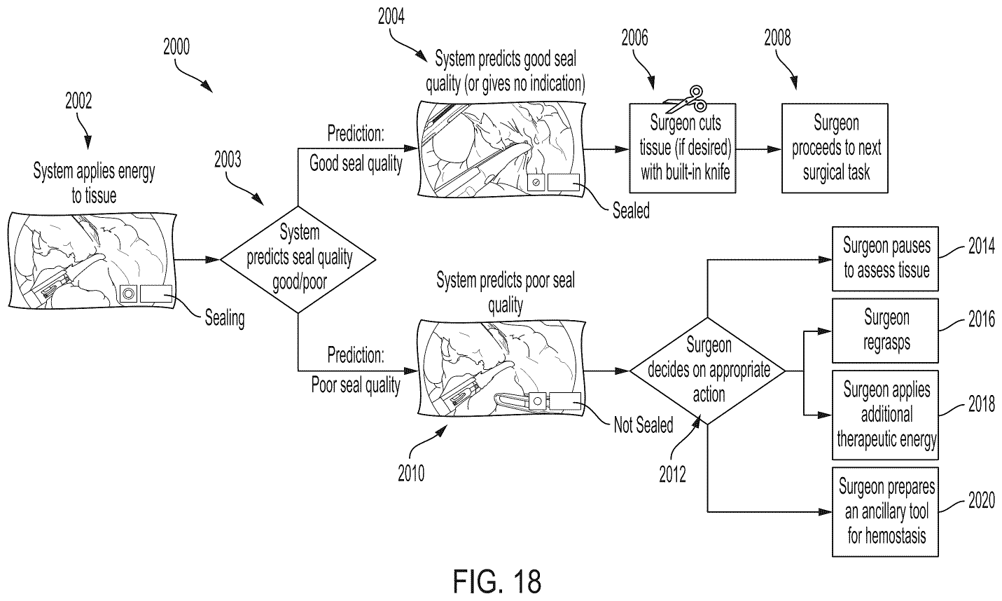

Referring now to FIG. 18, a flow chart 2000 is provided, according to at least one aspect of the present disclosure is provided. During a surgical procedure, the clamp jaw(s) of the electrosurgical tool, such as surgical instruments 1204, 1206, 1208, can grasp tissue 2002. The jaw(s) and the grasped tissue can be viewed on a display, such as display 135, display 711, or any other suitable display described elsewhere herein. Once the desired tissue has been grasped, a generator, such as generator module 140, RF energy source 794, or modular energy system 1000, or any other suitable energy generator described elsewhere herein, can provide RF energy to the electrode(s) of the electrosurgical tool, which can then apply the RF energy to the tissue to seal the same. Based on the application, a control system, such as processor module 132, processor 502, control circuit 760, or any other suitable control system described elsewhere herein, can predict 2003 the quality of the seal and determine an appropriate response, as will be described in more detail below. Based on the prediction, the control system can provide feedback to the surgeon, such as visual, audio, or haptic feedback, or combinations thereof, indicative of the type quality of the seal.

In one aspect, when the control system predicts 2004 that the seal is a good quality seal, the control system can provide feedback to surgeon indicating the same. Based on the good quality seal indication, the surgeon can confidently proceed with cutting 2006 the sealed tissue with a knife of the electrosurgical tool, such as with blade 768, ultrasonic blade 1249, ultrasonic blade 1228, or any other knife or blade described elsewhere herein. In various other embodiments, when the control system predicts 2004 that the seal is of good seal quality, the control system can provide no feedback to the surgeon. The lack of feedback can inform the surgeon that the electrosurgical tool performed as intended, i.e., that the applied seal was of good quality. The lack of feedback provides the advantage of preventing the surgeon from becoming distracted by feedback that does require a corrective action to be taken. Once the tissue has been cut by the blade, the surgeon can proceed 2008 to the next surgical task.

In one aspect, when the control system predicts 2010 that the seal is a poor quality seal, the control system can provide feedback to surgeon indicating the same. Based on the poor seal quality indication, the surgeon can decide 2012 on an appropriate course of action to take. In one aspect, the surgeon can pause 2014 the actuation of the electrosurgical tool to assess the sealed tissue prior to proceeding. In one aspect, the surgeon can release 2016 the tissue captured by the jaws of the electrosurgical tool and regrasp the tissue, or grasp different tissue. In one aspect, the surgeon can apply 2018 additional therapeutic energy to the grasped tissue, further sealing the tissue prior to proceeding. In one aspect, the surgeon can prepare 2020 an ancillary tool, such as a different energy instrument, that can be used to create hemostasis within the tissue.

### Data Collection and Annotation

As described above, to develop the algorithm that can implement the above-described predictor of seal quality, data is needed. In order to predict the quality of the seal, the control system can utilize a machine learning algorithm that is trained, optimized, and tested utilizing data (both input and output data) obtained from a variety of sources, such as clinical sources, preclinical sources, or benchtop sources, or combinations thereof. In one aspect, the more closely the data represents real use, the more variation that can be introduced during training of the algorithm, and the more robustly the algorithm will perform during use in a surgical procedure.

In some embodiments, the data used to train the algorithm can be obtained from clinical, real-world sources. In one aspect, the data can be clinical, real-world, data (both input and output data) that is obtained from identical or similar hardware components that are used on humans. Utilizing clinical data allows the algorithm to be trained with data that will very closely match what the algorithm with encounter during surgical procedures. In one aspect, the input data can include time-series electrical data from the electrosurgery generator, internal system event data, and device manufacturing data, as examples.

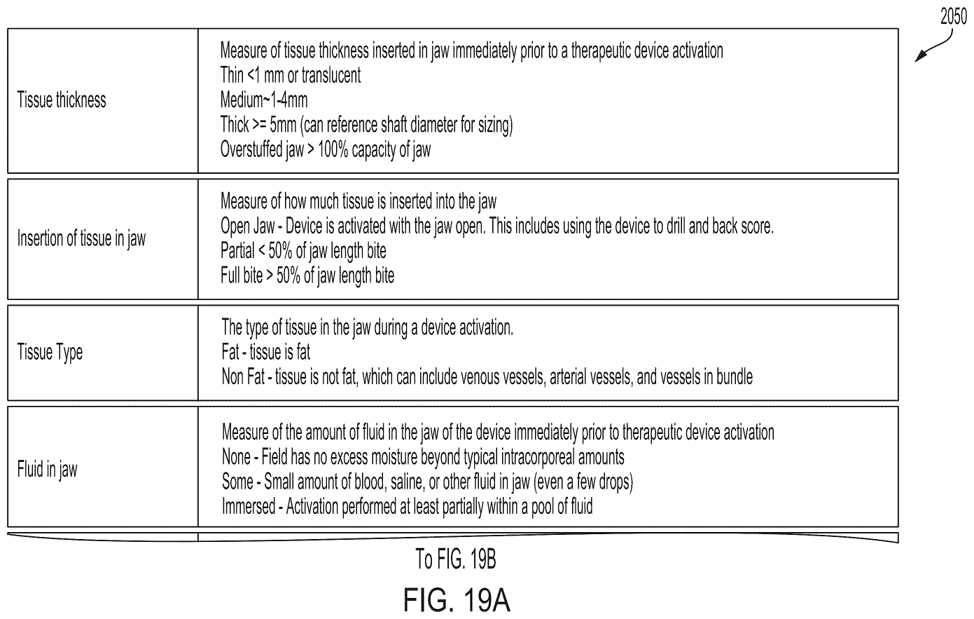

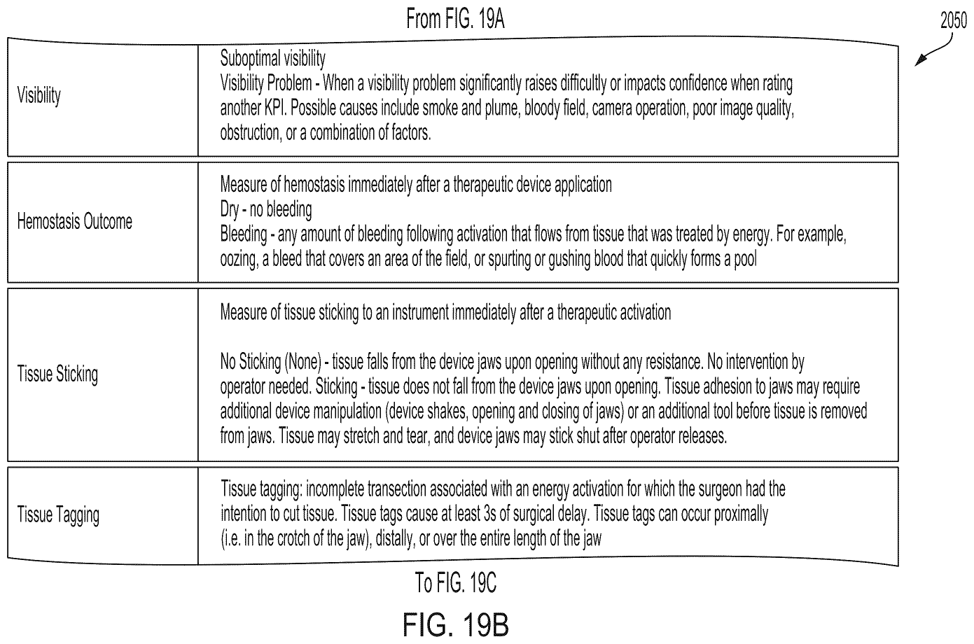

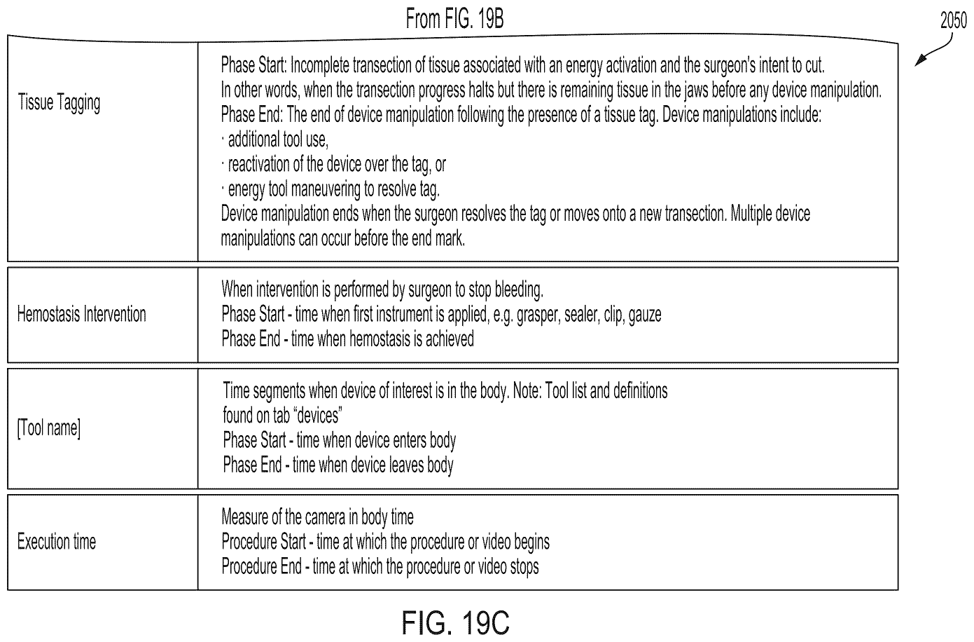

In various embodiments, the output data includes outputs obtained from surgical videos that are recorded and then annotated in post-processing. Video annotation is the process of applying labels to the surgical videos such that the algorithm can extract structured information from the videos. In one aspect, the labels, such as those shown in the table 2050 of FIGS. 19A-C, can be added to the videos by an operator based on the label definitions and the experience of the operator. In various embodiments, the labels can be stored in a memory, such as storage array 134, memory 504, or any other suitable memory described elsewhere herein. In various embodiments, the labels can be applied to the surgical videos by an operator at an input interface, such as a computer, a touchscreen monitor, or the like.

For seal quality prediction, it is important to know whether the tissue bleeds or not after every activation, i.e. the “Hemostasis Outcome”. Accordingly, in some embodiments, either “Bleeding” or “Dry” labels are applied to each activation, which serves as the ground truth for training algorithms. In some embodiments, bleeding vs. dry labels are converted to binary values for training the algorithm. In other embodiments, qualitative estimates of bleeding, e.g. dry, oozing, minor, major, can be encoded as ordinal data for multi-class algorithm training. Other labels obtained through video annotation, such as tissue thickness, tissue type, tissue sticking, as examples, can be used during training to aid in multi-task learning. In one aspect, multi-task learning is a machine-learning algorithm that is trained to predict multiple outputs, which improves the accuracy of seal quality prediction by taking advantage of additional tissue data not contained in the bleeding/dry labels.

In some embodiments, the data used to train the algorithm can be obtained from benchtop testing. Augmenting real-world data with benchtop tissue data enhances algorithm accuracy by expanding conditions outside of clinical use in humans. For example, creating benchtop data with devices with low and high tolerance components (at the edge of, or out of tolerance) enhances robustness of the algorithm by allowing it to learn to compensate better for these factors, and recognize the patterns of out-of-tolerance devices in the electrical data. This enhances accuracy of predictions even on nominal component tolerance devices.

In some embodiments, the data used to train the algorithm can be obtained from preclinical, live animal testing. Similar to adding benchtop testing data, described above, live animal data can further augment training and testing data sets. Adding animal data has the benefit of achieving good algorithm performance during development, and then pass verification and validation activities.

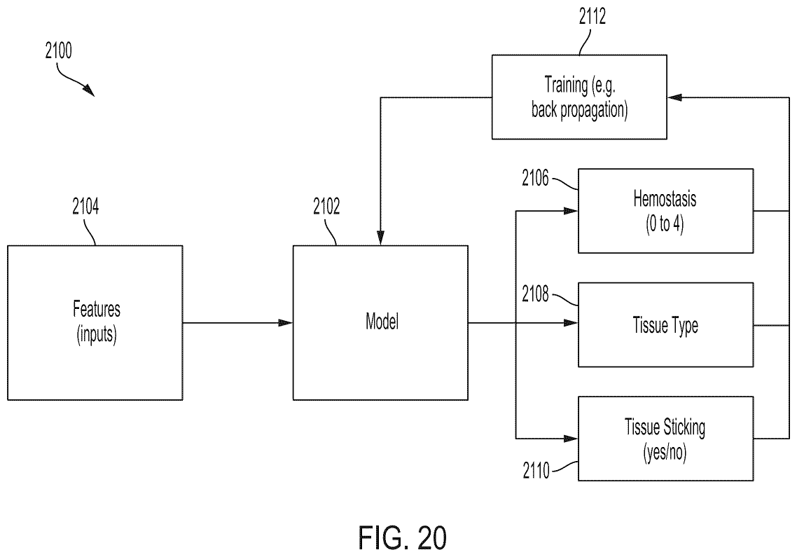

As referenced above, the machine-learning model can be trained to predict multiple outputs using multi-task learning to improve the accuracy of the seal quality prediction. Referring now to FIG. 20, a flowchart 2100 of multi-task learning during model training and validation is provided, according to at least one aspect of the present disclosure. The model 2102 can receive features (inputs) 2104, described in more detail below, and can output the hemostasis of the tissue 2106, the tissue type 2108, and if there was tissue sticking 2110. In various embodiments, these outputs, as described above, can include outputs obtained from surgical videos that are recorded and then annotated in post-processing. In various embodiments, the hemostasis of the tissue 2106 can be assigned a numerical value in a numerical range according to the quality of the seal. For example, the numerical range can include a minimum value, such as 0 and a maximum value, such as 4. These outputs can be fed to a training module 2112, which can then feed back into the model 2102 (i.e., back propagation) to further train the model 2102 and improve the accuracy of the seal quality prediction.

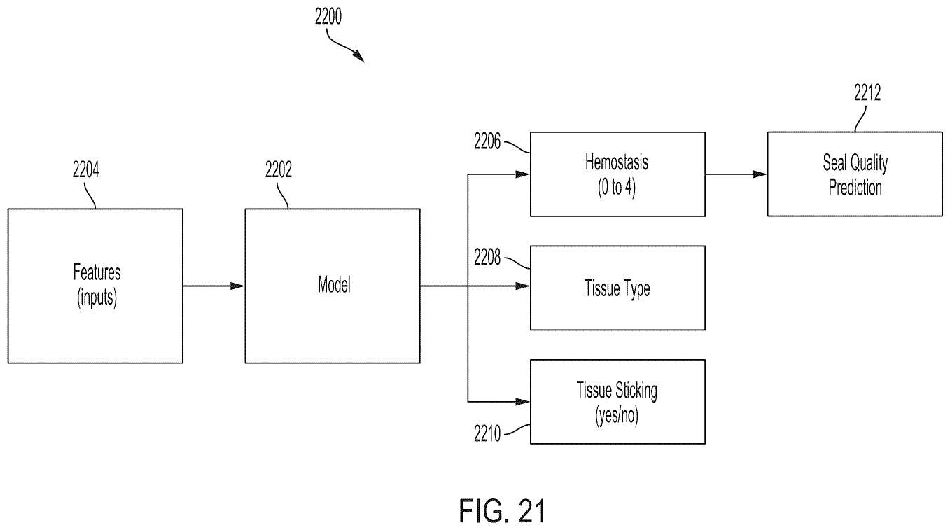

Referring now to FIG. 21, a flowchart 2200 of multi-task leaning during verification, validation, and clinical use is provided, according to at least one aspect of the present disclosure. The model 2202 can receive features (inputs) 2204, described in more detail below, and can output the hemostasis of the tissue 2206, the tissue type 2208, and if there was tissue sticking 2210. In various embodiments, the hemostasis of the tissue 2206 can be assigned a numerical value in a numerical range according to the quality of the seal. For example, the numerical range can include a minimum value, such as 0 and a maximum value, such as 4. According to the value, the system can make a prediction 2212 of the seal quality.

### Feature Generation

As referenced above, the algorithm model can receive features (inputs) that can be used to train the machine-learning algorithm.

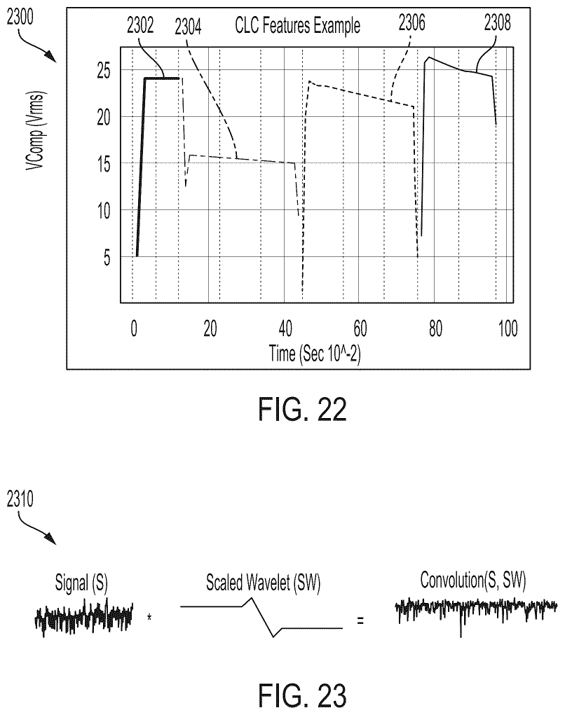

In various embodiments, the features can include algorithm-based features. In one aspect, the ABP generator algorithm can be controlled using a composite load curve (CLC) look-up table 2300, illustrated in FIG. 22. In various embodiments, the look-up table 2300 can be stored in a memory, such as memory, such as storage array 134, memory 504, memory 524, or any other suitable memory described herein. The given look-up table location, CLC code (section) and index (sub-section) values, during an activation is determined based on a combination of time and impedance. By segmenting electrical data based on what part of the control algorithm the activation is in, it can be determined how well the algorithm is performing.

For feature generation, the generator file is split up into CLC codes and then into smaller chunks inside of each CLC code. This can be seen in FIG. 22, where each section 2302, 2304, 2306, 2308 is a new CLC code number and the vertical lines show how each CLC code is further segmented. Summary statistics (max, min, mean, std, max/min ratio) are then taken from these smaller chunks and used as features for the machine learning model. This is done for a combination of electrical parameters time, voltage, current, impedance, power, and energy (t,V,I,Z,P,E). In various embodiments, global (the full activation) summary statistics are also used as features. In various embodiments, the amount of time spent in each CLC index is also used as features. The resultant vectors are then combined and stacked to create one vector for each activation.

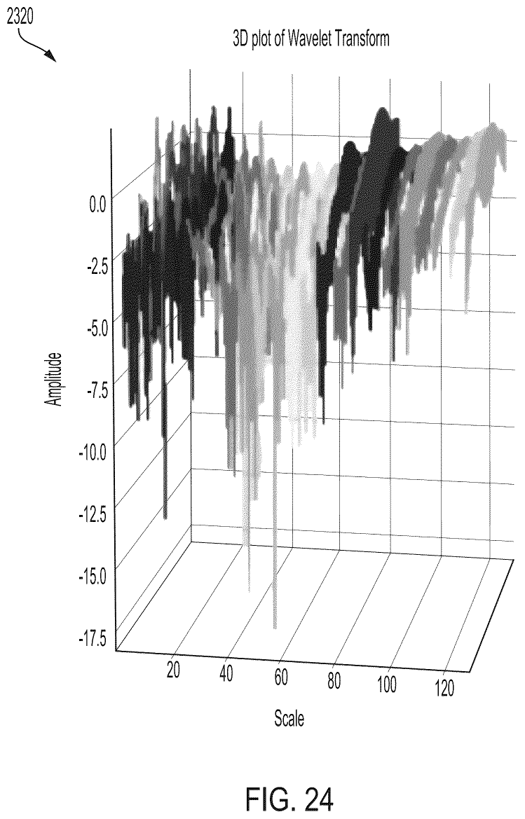

In various embodiments, the features can include wavelet-based features. In one aspect, wavelets (continuous and discrete) are a signal processing technique which takes a ‘mother’ wave, scales it, and translates it across a given signal. Unlike sine-waves which are not localized in time, wavelets are localized in time. This allows wavelet transformations to obtain time-information in addition to frequency information. Since the wavelet is localized in time, a signal can be multiplied with the wavelet at different locations in time, this procedure being also known as a convolution, as can be seen in the representation 2310 shown in FIG. 23. After this has done for the original (mother) wavelet, it can be scaled such that it becomes larger and repeat the process. The resulting transformation 2320, shown in FIG. 24, is like a Fast Fourier Transform (“FFT”); however, it provides context of the signal in both frequency and time domains.

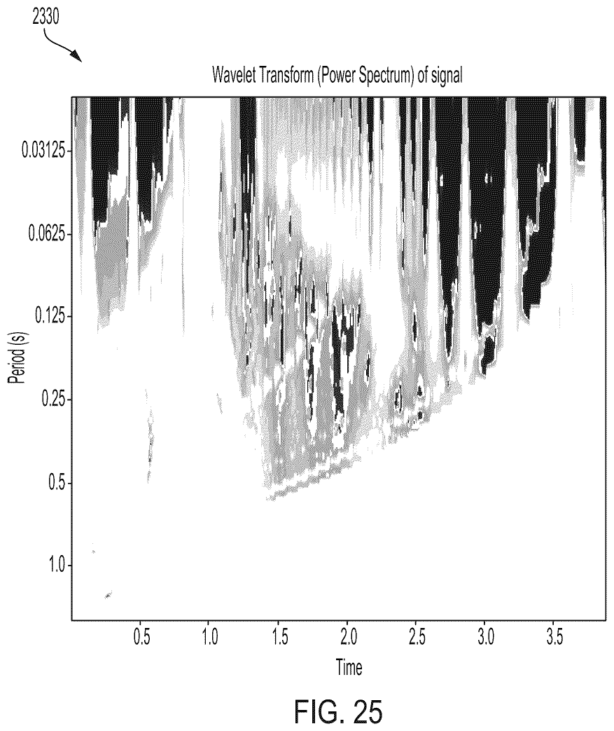

As referenced above, the wavelets can include discrete wavelets and continuous wavelets. Discrete wavelet transformations (“DWT”) and continuous wavelet transformations (“CWT”) are taken of the raw signal and used as features. In some embodiments, for both DWT and CWT, the mother wavelet used is ‘db4’. In some embodiments, for the CWT, 128 scales of the mother wavelet are used, the resultant coefficients and frequencies of the transformation are stacked and zero-padded to create even length feature arrays. In some embodiments, for DWT, summary statistics and entropy calculations of the resultant coefficients are used instead of the pure coefficients. An example of a wavelet transform (power spectrum) of signal is shown on graph 2330 in FIG. 25.

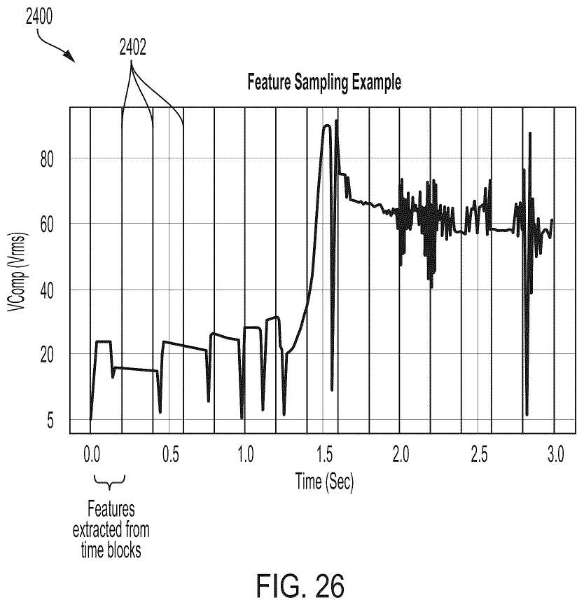

In various embodiments, the features can include time-based features. Referring to graph 2400 shown in FIG. 26, the ABP electrical parameters voltage, current, impedance, power, and energy (V,I,Z,P,E) can be split into ‘x’ time length segments 2402. Summary statistics (max, min, mean, std, max/min ratio) are then taken from these segments. The resultant vectors are then stacked to create one vector, as shown in graph 2400.

In various embodiments, the features can include raw signal features. The raw signal from the generator can be parsed into 5 vectors—voltage, current, impedance, power, and energy (V,I,Z,P,E) —which can then be stacked on top of each other and zero-padded, zeros are added to then end of each signal, to ensure that each vector for every activation is the same length. In one aspect, there can be 540 numbers per signal, where 540 is the theoretical maximum length of an activation (5.4 seconds).

### Machine Learning (“ML”) Models

As referenced above, preprocessing may result in hundreds or thousands of features that are fed into the machine learning models, which takes the inputs and generates a numeric prediction. In one aspect, with machine leaning (“ML”) models, the main idea of a sampling-based approach is to modify the distribution of events so that the rare class is well represented in the training sample. Given that bleeding cases represent only ˜10% of all cases, various class balancing techniques are applied to the machine learning model.

In the case of undersampling, a random sample is taken from the majority class, i.e., non-bleeding events. A potential problem with undersampling, however, is that some of useful non-bleeding instances may not be chosen for training and the classifier will not be optimal. In the case of oversampling, the replication of events are taken from the minority class, i.e., bleeding cases. A potential problem with oversampling, however, is that this technique results into overfitting for noisy data which will be replicated multiple times. This results into poor model generalization.

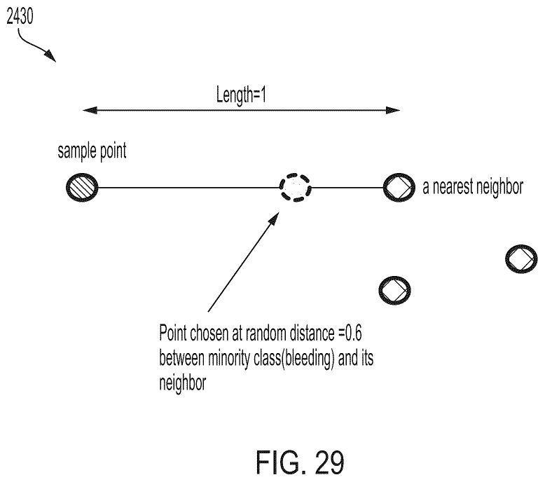

Accordingly, the present disclosure provides a hybrid approach of oversampling and undersampling, referred to herein as Synthetic Minority Oversampling Technique (“SMOTE”), which creates artificial minority class data using features space similarities. SMOTE is described in more detail in the paper titled “SMOTE: Synthetic Minority Over-sampling Technique”, by Notesh Chawla et al., which published in June 2002, which is hereby incorporated by reference in its entirety herein. The advantages of SMOTE are that it alleviates overfitting caused by random oversampling as synthetic examples are generated rather than replication of instances. Furthermore, there is no loss of information. In one aspect, referring to representation 2430 shown in FIG. 29, SMOTE can be implemented by taking the difference between a sample point and one of its nearest neighbors, multiply the difference by a random number between 0 and 1, and add the multiplied number to the feature vector. This causes the selection of a random point along the line segment between two specific features.

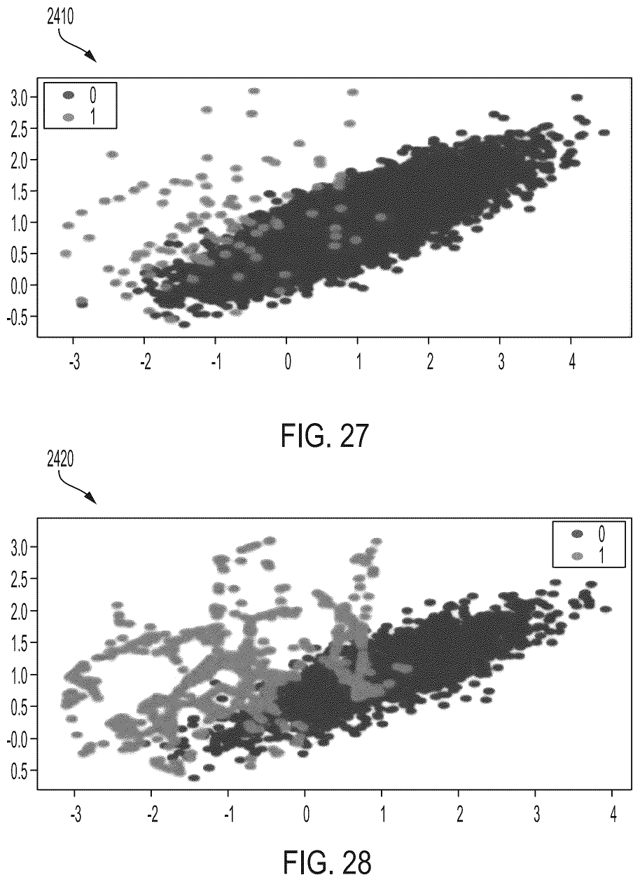

Referring now to FIG. 27, a scatter plot 2410 of a dataset is provided showing the large mass of points that belong to the majority class (dark dots, “0”) and a small number of points spread out for the minority class (light dots, “1”). The minority class is oversampled and the majority class is undersampled using SMOTE then plotted, which results in scatter plot 2420 shown in FIG. 28.

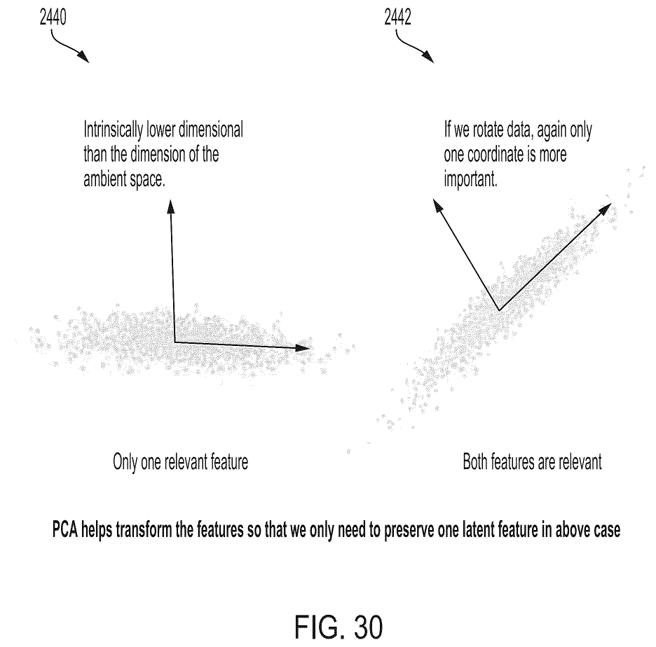

224 summary statistics were obtained using multiple approaches involving time based, algorithmic, wavelet, and raw features. Principal Component Analysis (“PCA”) was performed for extracting hidden (potentially lower dimensional) structure from the high dimensional feature space. PCA is an orthogonal projection or transformation of the data into a (possibly lower dimensional) subspace so that the variance of the projected data is maximized. Identifying the axes is known as Principal Components Analysis, and can be obtained by using classic matrix computation tools (Eigen or Singular Value Decomposition). Reference is made to FIG. 30 where PCA is shown to transform the features from one state 2440 to a second state 2442 such that only latent feature needs preserved.

In some aspects, for ML Models, a prediction model can be used that include a mix of linear and tree based classifiers. The model framework had an input of a dataframe of size [n,224] where 224 represents summary statistics for electric signals obtained from feature engineering. In one aspect, preprocessing is accomplished with SMOTE or PCA, as examples, both of which are described above. Tissue classification included bleeding or dry.

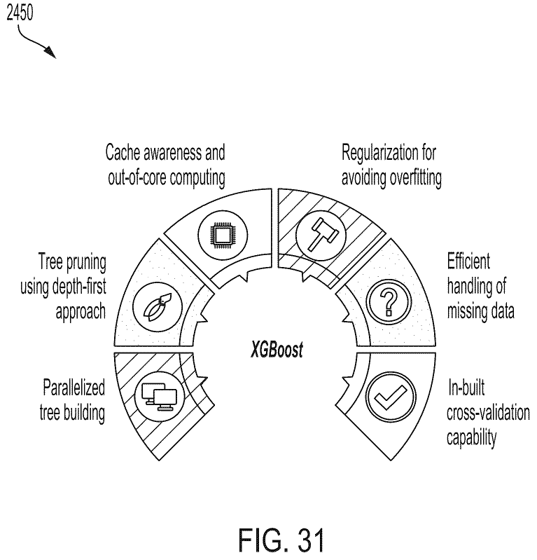

For logistic regression, a binary classification model that uses logistic (sigmoid) function to model probabilities was used that includes a regularized model with alpha being equal to 0.5 and beta being equal to 0.5. Hyperparameter tuning was utilized to obtain optimal values for alpha and beta. A support vector machine (“SVM”) algorithm was used to find a hyperplane in an N-dimensional space (N—the number of features) that distinctly classifies the data points. The linear SVM was fit with L2 regularization. Radial Kernel functions were utilized to model non-linearities. Referring to FIG. 31, an Xgboost 2450 was utilized, which is a machine learning library built around an efficient implementation of booting for tree models (like GBM). Xgboost allows model tuning back on multiple parameters, such as number of trees (300), the max depth (3, 6, 8), the metric (binary Logloss), L1 regularization (0, 1, 2), L2 regularization (1, 2), number of iterations (100, 150, 250), and boosting types (‘Dart’, ‘gbtree’). More information regarding XGBoost is described in the paper entitled “Comparative Analysis of Artificial Neural Network and XGBoost Algorithm for PolSAR Image Classification”, by Nimra Menon et al., which published in November 2019, which is hereby incorporated by reference in its entirety herein.

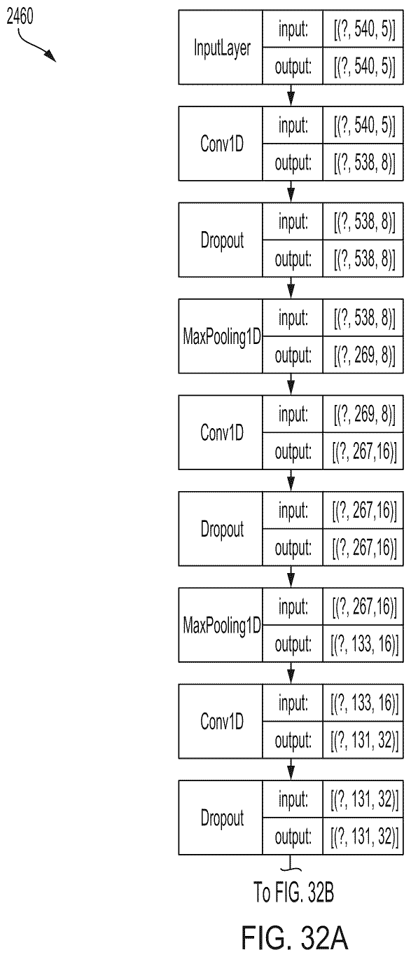

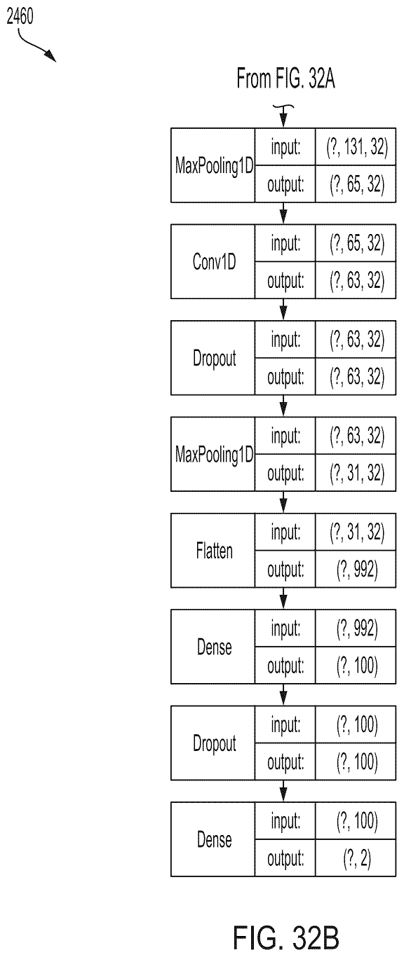

Other models used were CNN models, which are deep learning classification models that can be trained to classify patterns by extracting, through learnable weights and biases that describe multiple layers of convolution, informative aspects of the signal. In one aspect, a 1D CNN model can include an input of Five [1×540] vectors that describe the electrical signal. Preprocessing can be [1×7] median filter, standard scaling. Referring to FIGS. 32A and 32B, the model architecture 2460 can include Conv1D (8)>Dropout(0.2)>Max(2)>Conv1D (16)>Dropout)(0.2)>Max(2)>Conv1D(32)>Dropout(0.2)>Max(0.2)>Conv1D(32)>Dropout(0.2)>Max(0.2)>Flatten>Dense(100)>Dropout(0.2)>Dense(2).

Other models used were CNN+Dense multihead architecture models, which are deep learning classification models that can be trained to classify patterns by extracting, through learnable weights and biases that describe multiple layers of convolution, informative aspects of the signal, and combining this information with other inputs. These other inputs are processed by a neural network with a number of densely connected layers. Outputs from multiple heads are combined and processed by a final stack of dense layers.

In one aspect, the model can include an input of two [1×540] vectors that describe the electrical signal and One [1×5] vector describing the mechanical parameters. Preprocessing can be [1×7] median filter, standard scaling for the electrical signal and standard scaling for the mechanical parameter vectors.

### Metrics

As described above, the threshold can be chosen for a particular model to appropriately balance the sensitivity and specificity of the prediction. To optimize the efficacy of the solution in the context of surgical workflow, it is important to balance out false positives (i.e. false alarms leading to alarm fatigue) as well as false negatives (i.e. missed bleeding events leading to bleeding).

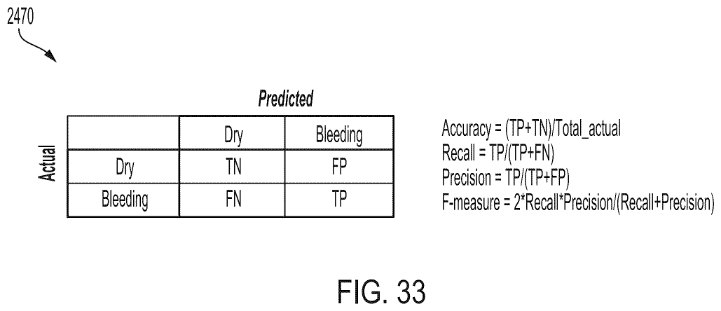

Referring now to FIG. 33, a confusion matrix table 2470 is provided according to at least one aspect of the present disclosure that can be used to compare a predicted outcome to an actual outcome. In one aspect, the confusion matrix can be utilized in training the ML algorithm. For instance, in the event that it is predicted that the tissue with be dry and is actually dry, a true negative designation ‘TN’ is provided. In the event that it is predicted that the tissue with be dry and is actually bleeding, a false negative designation ‘FN’ is provided. In the event that it is predicted that the tissue with be bleeding and is actually dry, a false positive designation ‘FP’ is provided. In the event that it is predicted that the tissue with be bleeding and is actually bleeding, a true positive designation ‘TP’ is provided.

Accuracy of the system is determined by the proportion of true results among the total number of cases examined. The equation for accuracy is provided as the number of TP and TN designations divided by the number of total actual number of cases. Given that the data is unbalanced in this case (approximately 10% bleeding events), accuracy fails to correctly capture precision and sensitivity values for bleeding events.

The proportion of predicted positives against those that are truly positive determines precision of the system. The equation for precision is provided as the number of TP designations divided by the sum of the TP and FP designations. In this case, the classifier showed high precision values, however, it failed to predict all bleeding.

The proportion of actual positives corrected classified determines the recall of the system. The equal for recall is provided as the number of TP designations divided by the sum of TP and FN designations. Optimizing classifier model based on this metric resulted in low precision scores.

An F1 score helps obtain recall precision scores within clinical viability threshold. The F1 score is a number between 0 and 1 and is the harmonic mean of precision and recall. The equation for F1 score is provided as 2*Recall*Precision/(Recall+Precision).

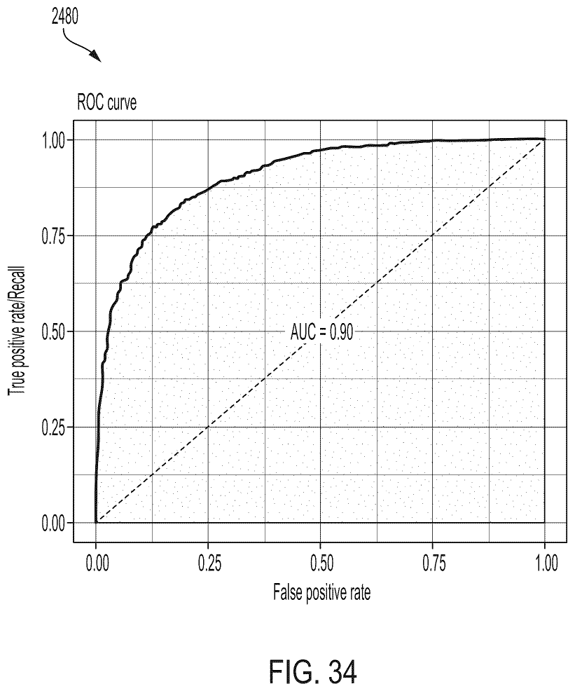

An Area under Curve (“AUC”) receiver operating characteristic (“ROC”) curve indicates how well the probabilities from the positive classes are separated from the negative classes. It is plotted between True Positive Rate and False Positive Rate values, as shown in example graph 2480 in FIG. 34. Given that the dataset is skewed, AUC ROC curve continues to score high despite low accuracy with minority class. For example, if the model overfits the majority class, it might fail to predict minority class but still result in high true positive rate (“TPR”) and low false positive rate (“FPR”) values. This results into high AUC Scores.

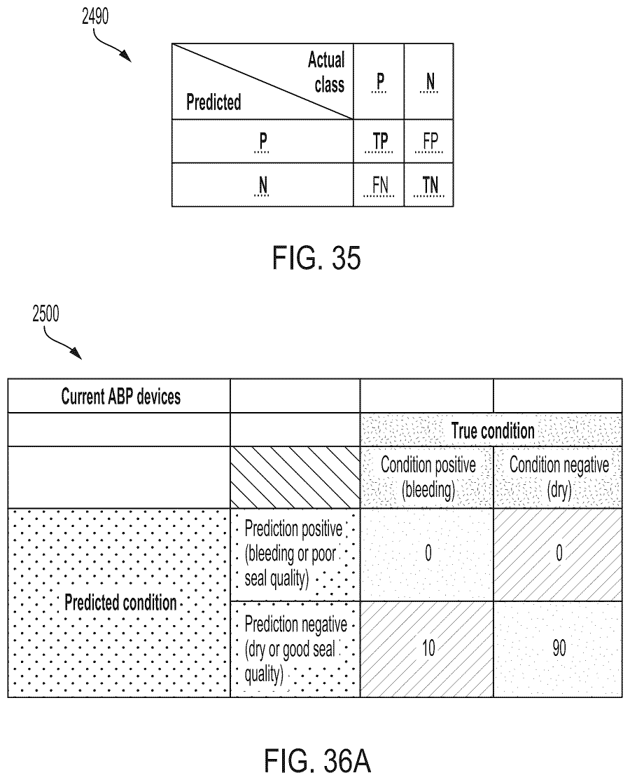

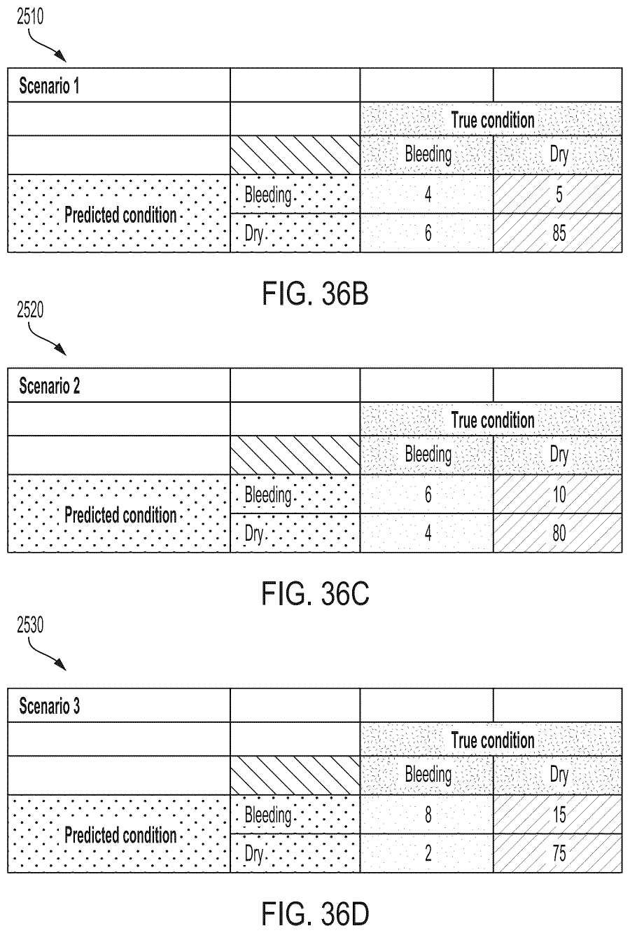

Target metrics were developed in conversations with various surgeons across North America and APAC. While discussing acceptable false positive (alarm fatigue) and false negative rates (missed bleeding events) with surgeons, a confusion matrix 2490 shown in FIG. 35, was used to help facilitate discussion and reach a numerical target value. After the overall concept of seal quality predictions was explained to surgeons, it was explained that there was a trade-off between false positive and false negative rates for a given machine learning model. Surgeons were then walked through the 4 confusion matrices 2500, 2510, 2520, 2530, shown in FIGS. 36A-36D, with various false positive and false negative rates and asked to give their opinion of each and which scenario was preferred.

After all the discussions were complete, the responses were aggregated to determine a clinical viability threshold. It was determined that the tolerance for “alarm fatigue”, i.e. false positives, was roughly 2-4× higher than “missed bleeding events”, i.e. false negatives.

### Integration and Interface

As referenced above, information must be communicated to the surgeon so they can decide on the appropriate course of action once the quality of the seal has been predicted. Communicating the prediction of seal quality can be done in many ways, and must strike the balance between being distracting or obtrusive to the surgeon, but should also minimize the likelihood of missing information.

In one aspect, it would be advantageous to display a non-obtrusive message on a monitor, such as monitor 14, a primary lap monitor, or any other monitor or display described elsewhere herein, when a poor seal is predicted, and to display nothing when a good seal is predicted. Considering good seals occur most of the time, not displaying seal quality information in these instances would help minimize information overload and distraction.

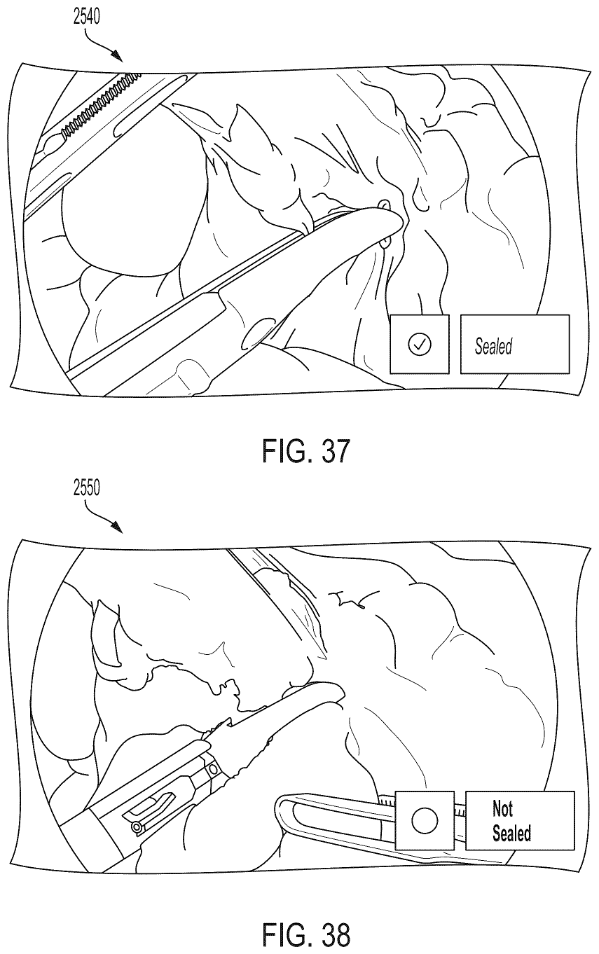

In various embodiments, visual methods (on-screen displays 2540, 2550, such as those shown in FIGS. 37 and 38) of displaying poor seal quality comprise some or all of binary feedback (good vs bad, or sealed vs. not sealed), continuous feedback (Seal quality percent, 0-100%, which can be accompanied by a recommended threshold indicated visually by a color change), and/or discrete feedback (red, yellow, green indicator icon).

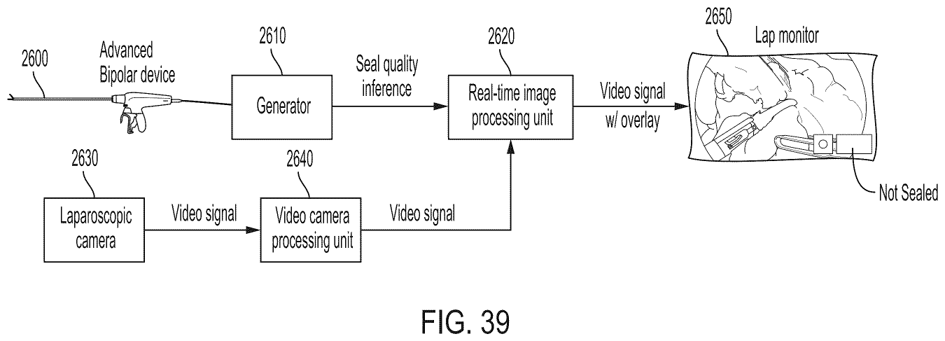

A first embodiment of a seal quality prediction system for an advanced bipolar device 2600 is provided in FIG. 39 where the seal quality inference occurs within an advanced bipolar generator 2610, which is similar to generator module 140, energy source 794, or modular energy system 1000, or any other generator described elsewhere herein. The inference output is communicated to a real-time image processing unit 2620, which is similar to processor module 132, processor 502, control circuit 760, or any other suitable image processing unit described elsewhere herein, which overlays seal quality information onto the laparoscopic video image 2650 which is captured using a laparoscopic camera 2630 and a video camera processing unit 2640. The advantage of this arrangement is that this would be compatible with any 3rd party laparoscopic video system.

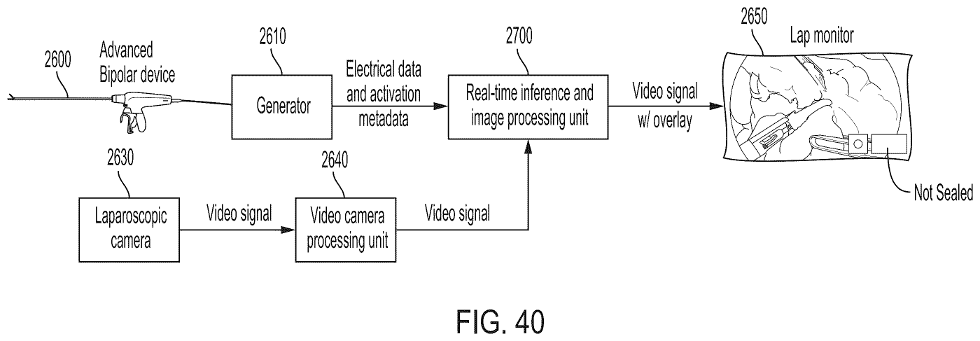

A second embodiment of a seal quality prediction system for an advanced bipolar device 2600 is provided in FIG. 40. The second embodiment is similar to the first embodiment, described above, except that the advanced bipolar generator 2610 sends raw electrical data and activation metadata to a real-time inference and image processing unit (RTIIP) 2700 instead of to real-time image processing unit 2620. The seal quality inference occurs within the RTIIP 2700, which then overlays seal quality information onto the laparoscopic video image. The advantage of this arrangement is that the RTIIP 2700 could be provided as a stand-alone product compatible with the energy system (generator and device). The inference feature could be provided as a software add-on. This may also obviate the need for design changes to the energy system, which may have regulatory and R&D expense advantages.

In various embodiments, the system can also provide audible means for communicating seal quality information. In some embodiments, an audio feedback module, such as any suitable audio feedback module described elsewhere herein, can emit a first sound effect during the sealing phase of device activation, a secondary sound effect in case of a high quality seal, and a third sound effect for a poor quality seal. Various other embodiments are envisioned where no sound effect is emitted in the case of a high seal quality so as to not distract the surgeon. Rather, the audio feedback module only provides feedback in the event of a poor quality seal.

In various embodiments, the energy device, such as instrument 112, surgical instrument 790, or surgical instruments 1204, 1206, 1208 can be designed with a haptic device, such as a piezoelectric motor, which buzzes or clicks upon a poor seal quality prediction. Various embodiments are envisioned where the haptic device provides haptic feedback in the event of a high quality seal. Various other embodiments are envisioned where the haptic device does not provide haptic feedback in the event of a high quality seal so as to not distract the surgeon.

The entire disclosures of U.S. Pat. Nos. 10,624,691, 10,842,523, 11,291,510, 11,311,342, 11,259,830, 11,304,699, 11,109,866, 11,298,129, 11,229,437, 11,241,235 and U.S. Patent Application Publication Nos. 2019/0206562, 2019/0200981, 2019/0208641, 2019/0201594, 2019/0201045, 2019/0200844, 2019/0201136, 2019/0206569, 2019/0201137, 2019/0125459, 2019/0125458, 2019/0125455, 2019/0125454, 2019/0274706, 2019/0201046, 2019/0201047, 2019/0104919, 2019/0125361, 2019/0200977, 2019/0298350, 2019/0206564, 2019/0206565, 2020/0100830, 2020/0078070, 2020/0078076, 2020/0078106, 2020/0100825, 2021/0196334, 2021/0196354, 2021/0196302, 2020/0345353, 2022/0031315 are hereby incorporated by reference in their entireties herein.

Although various devices have been described herein in connection with certain embodiments, modifications and variations to those embodiments may be implemented. Particular features, structures, or characteristics may be combined in any suitable manner in one or more embodiments. Thus, the particular features, structures, or characteristics illustrated or described in connection with one embodiment may be combined in whole or in part, with the features, structures or characteristics of one ore more other embodiments without limitation. Also, where materials are disclosed for certain components, other materials may be used. Furthermore, according to various embodiments, a single component may be replaced by multiple components, and multiple components may be replaced by a single component, to perform a given function or functions. The foregoing description and following claims are intended to cover all such modification and variations.

The devices disclosed herein can be designed to be disposed of after a single use, or they can be designed to be used multiple times. In either case, however, a device can be reconditioned for reuse after at least one use. Reconditioning can include any combination of the steps including, but not limited to, the disassembly of the device, followed by cleaning or replacement of particular pieces of the device, and subsequent reassembly of the device. In particular, a reconditioning facility and/or surgical team can disassemble a device and, after cleaning and/or replacing particular parts of the device, the device can be reassembled for subsequent use. Those skilled in the art will appreciate that reconditioning of a device can utilize a variety of techniques for disassembly, cleaning/replacement, and reassembly. Use of such techniques, and the resulting reconditioned device, are all within the scope of the present application.

The devices disclosed herein may be processed before surgery. First, a new or used instrument may be obtained and, when necessary, cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, and/or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta radiation, gamma radiation, ethylene oxide, plasma peroxide, and/or steam.

While this invention has been described as having exemplary designs, the present invention may be further modified within the spirit and scope of the disclosure. This application is therefore intended to cover any variations, uses, or adaptations of the invention using its general principles.

The foregoing detailed description has set forth various forms of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, and/or examples can be implemented, individually and/or collectively, by a wide range of hardware, software, firmware, or virtually any combination thereof. Those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, the term “control circuit” or “control system” may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

As used in any aspect herein, an “algorithm” refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “to,” “configurable to,” “operable/operative to,” “adapted/adaptable,” “able to,” “conformable/conformed to,” etc. Those skilled in the art will recognize that “to” can generally encompass active-state components and/or inactive-state components and/or standby-state components, unless context requires otherwise.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

The term “substantially”, “about”, or “approximately” as used in the present disclosure, unless otherwise specified, means an acceptable error for a particular value as determined by one of ordinary skill in the art, which depends in part on how the value is measured or determined. In certain embodiments, the term “substantially”, “about”, or “approximately” means within 1, 2, 3, or 4 standard deviations. In certain embodiments, the term “substantially”, “about”, or “approximately” means within 50%, 20%, 15%, 10%, 9%, 8%, 7%, 6%, 5%, 4%, 3%, 2%, 1%, 0.5%, or 0.05% of a given value or range.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

What is claimed is:

1. A surgical device, comprising:
an end effector comprising:
a first jaw; and
a second jaw;
a first sensor to detect tissue disposed between the first jaw and the second jaw;
a second sensor to detect the end effector in a closed configuration;
a third sensor to detect motion of the end effector; and
a control circuit communicably coupled to the first sensor, the second sensor, and the third sensor, the control circuit comprising a processor and a memory, wherein the memory stores instructions that, when executed by the processor, cause the control circuit to:
determine that the end effector is in a closed configuration based on first sensor data;
determine presence of tissue disposed between the first jaw and the second jaw based on second sensor data;
monitor motion of the end effector in the closed configuration with tissue present between the first and second jaw based on third sensor data;
detect motion of the end effector outside of a predetermined range based on the motion; and
provide feedback data based on the detected motion of the end effector.

2. The surgical device of claim 1, wherein the memory stores further instructions that, when executed by the processor, cause the control circuit to calculate tension on the tissue based on the motion.

3. The surgical device of claim 1, wherein the feedback is visual.

4. The surgical device of claim 3, wherein the visual feedback is superimposed over a display image of a surgical site.

5. The surgical device of claim 1, wherein the feedback is auditory.

6. The surgical device of claim 1, wherein the first jaw comprises a clamp arm and the second jaw comprises an ultrasonic blade.

7. The surgical device of claim 1, wherein the first jaw comprises an anvil and the second jaw comprises a staple cartridge.

8. A surgical device, comprising:
an end effector comprising:
a first jaw; and
a second jaw;
a first sensor to detect tissue disposed between the first jaw and the second jaw;
a second sensor to detect that the end effector is in a closed configuration;
a first fiducial mark;
a second fiducial mark; and
a control circuit communicably coupled to the first sensor, the second sensor, and a camera, the control circuit comprising a processor and a memory, wherein the memory stores instructions that, when executed by the processor, cause the control circuit to:
receive video data of a surgical site from the camera;
determine that the end effector is in the closed configuration on first sensor data;
determine presence of tissue disposed between the first jaw and the second jaw based on second sensor data;
determine a location of a device tip in the video data based on the first fiducial mark and the second fiducial mark;
determine a region of interest in the video data based on the location of the device tip in the video data;
analyze the region of interest of the end effector in the closed configuration with tissue present between the first jaw and the second jaw;
determine tension on the tissue based on the analysis; and
provide feedback based on the tension.

9. The surgical device of claim 8, wherein the memory stores further instructions that, when executed by the processor, cause the control circuit to detect motion of the end effector outside of a predetermined range based on the analysis.

10. The surgical device of claim 8, wherein the memory stores further instructions that, when executed by the processor, cause the control circuit to determine device type based on the first fiducial mark and the second fiducial mark.

11. The surgical device of claim 8, wherein the feedback is visual.

12. The surgical device of claim 11, wherein the visual feedback is superimposed over a display image of a surgical site.

13. The surgical device of claim 8, wherein the feedback is auditory.

14. The surgical device of claim 8, wherein the first jaw comprises a clamp arm and the second jaw comprises an ultrasonic blade.

15. The surgical device of claim 8, wherein the first jaw comprises an anvil and the second jaw comprises a staple cartridge.

16. A surgical system, comprising:
a surgical instrument comprising an end effector to capture tissue, wherein the end effector comprises an electrode to apply radio-frequency (RF) energy to the tissue captured by the end effector;
an RF energy source to provide RF energy to the electrode; and
a control circuit, to:
transmit a control signal to the RF energy source, wherein the control signal causes the RF energy source to provide RF energy to the electrode to apply a seal to the tissue captured by the end effector;
predict a quality of the seal; and
provide feedback to a user based on the prediction.

17. The surgical system of claim 16, wherein to predict the quality of the seal, the control circuit is to:
generate a value associated with the seal; and
compare the value to a seal threshold;
wherein to provide feedback to the user based on the prediction, the control circuit is to provide feedback to the user based on results of the compare.

18. The surgical system of claim 17, wherein the control circuit is to abstain from providing feedback based on the value reaching or exceeding the seal threshold.

19. The surgical system of claim 17, wherein the surgical system further comprises a display, wherein the control circuit is to transmit a signal to the display based on the value being below the seal threshold, wherein the feedback comprises visual feedback on the display, and wherein the visual feedback is based on the signal.

20. The surgical system of claim 17, wherein the surgical system further comprises an audio feedback module, wherein the control circuit is to transmit a signal to the audio feedback module based on the value being below the seal threshold, wherein the feedback comprises audio feedback via the audio feedback module, and wherein the audio feedback is based on the signal.

21. The surgical system of claim 17, wherein the surgical system further comprises a haptic feedback module, wherein the control circuit is to transmit a signal to a haptic feedback module based on the value being below the seal threshold, wherein the feedback comprises haptic feedback via the haptic feedback module, and wherein the haptic feedback is based on the signal.

22. The surgical system of claim 16, wherein the RF energy source comprises the control circuit.

23. The surgical system of claim 16, wherein the surgical system further comprises a processing unit comprising the control circuit.
